(19) United States
(12) Patent Application Publication
(10) Pub. No.: US 2020/0113910 A1
(43) Pub. Date: Apr. 16, 2020

(54) TRIAZINETRIONE DERIVATIVES AND THEIR USE AS MODULATORS OF NEUROTROPHIN RECEPTOR AND RECEPTOR TYROSINE KINASES

(71) Applicant: ALZECURE PHARMA AB, Huddinge (SE)

(72) Inventors: Gunnar NORDVALL, Rönninge (SE); Pontus FORSELL, Huddinge (SE); Johan SANDIN, Stockholm (SE)

(21) Appl. No.: 16/471,923

(22) PCT Filed: Dec. 21, 2017
(86) PCT No.: PCT/GB2017/053868

§ 371 Date: Jun. 20, 2019

(30) Foreign Application Priority Data
Dec. 21, 2016 (SE) 1651706-2

(51) Int. Cl. A61K 31/53

(52) U.S. Cl. CPC A61K 31/53; A61K 45/06

(57) ABSTRACT

The present invention relates to anew use of 4-phenoxy-phenyl-1,3,5-triazine derivatives or pharmaceutically acceptable salts thereof, according to formula I, wherein R¹, R² and U have meanings as provided in the description, as medicaments for the treatment and/or prevention of diseases characterised by impaired signalling of neurotrophins and/or other trophic factors. In particular, the invention relates to the treatment of such diseases in patients with the Val66Met mutation in the brain-derived neurotrophic factor gene.

### FIELD OF THE INVENTION

The invention relates to a new use of 4-phenoxy-phenyl-1,3,5-triazine derivatives, and pharmaceutically acceptable salts thereof, as medicaments for the treatment and/or prevention of diseases characterised by impaired signalling of neurotrophins and/or other trophic factors. The invention also relates to novel compounds, pharmaceutical compositions and the uses of the same in the treatment and/or prevention of diseases characterised by impaired signalling of neurotrophins and/or other trophic factors.

### BACKGROUND

Nerve growth factor (NGF), Brain Derived Neurotrophic Factor (BDNF) and neurotrophin-3 (NT-3) and neurotrophin-4/5 all belong to the neurotrophin protein family. These hormones act through a class of receptor tyrosine kinases called tropomyosin-receptor kinase (Trk). Ligand binding to Trks initiates receptor dimerization and autophosphorylation of the kinase domain, which activates the kinase activity of the receptor. This results in further receptor phosphorylation at Tyr490, Tyr751 and Tyr785 of TrkA (or their equivalent residues in other Trk receptors). This phosphorylation leads to adaptor binding sites that couple the receptor to SHC adaptor protein 1 (SHC-1), phosphoinositide 3-kinase (PI3K) and phospholipase Cγ1 (PLCγ1). The coupling of adaptor proteins to the receptor initiates several different cellular events leading to e.g. neurite outgrowth and axonal elongation. These receptors, and their signalling pathways, play a pivotal role in many key processes in the brain e.g. hippocampal neurogenesis, synaptic plasticity, and long-term potentiation, a proposed mechanism underlying memory formation at the level of the synapse. Both NGF/TrkA and BDNF/TrkB-stimulated signalling is also necessary for the survival and morphogenesis of neurons.

In addition to activation of Trk-receptors by classical ligand binding, there are ligand independent events that can regulate neurotrophin signalling.

The balance between the activity of the receptor tyrosine kinase and the activity of tyrosine phosphatases intricately regulates the levels of phosphorylated receptor. Thus, protein tyrosine phosphatases such as PTP-1B or other phosphatases can increase neurotrophin signalling and regulate temporal and spatial activity of the Trk-receptor as well as receptor tyrosine kinases.

Also, adenosine and adenosine agonists can mediate phosphorylation of Trk-receptors, via a mechanism that requires the adenosine 2A (A2A) receptor. This phosphorylation of Trk-receptors is independent of ligand binding suggesting that modulation of Trk-receptor signalling can be accomplished by several different mechanisms.

Other key members of growth factor family are the fibroblast growth factors (FGF 1-23) and insulin growth factors (IGF 1-2). FGFs, through binding to their receptors (FGFR1, FGFR2, FGFR3, and FGFR4), play a key role in proliferation and differentiation processes of a wide variety of cells and tissues and thereby are involved in processes such as angiogenesis, wound healing, embryonic development and various endocrine-signalling pathways. IGF on the other hand, has a similar molecular structure to insulin, and binds to its receptor IGF-1R mediating effects on growth in childhood and continuing to have anabolic effects in adults. Both of these factors have also been implicated in the pathogenesis of neurodegenerative disorders of the central nervous system (CNS) such as Alzheimer's disease (Li J S et al., Med Hypotheses, 2013 Apr. 80(4), 341-4 and Gasparini et al., Trends Neurosci. 2003 Aug. 26(8):404-6).

Synapse loss and a decrease in the hippocampal volume are pathological signatures of Alzheimer's disease in the brain and a number of studies suggest that synapse loss is the best neuroanatomical indicator of cognitive decline in the disease. Basal forebrain cholinergic neurons (BFCN) are a subpopulation of neurons that seem to be particularly vulnerable to the pathology of AD. Dysfunctional atrophy of these neurons, which in turn results in severe loss of cortical and hippocampal innervation, may be the source for the malfunction of the cholinergic system in AD (Bartus R T Exp Neurol 2000;163:495-529). The severe cortical cholinergic deficits in the disease also include a loss of choline acetyltransferase (ChAT) and acetylcholinesterase (AChE) activity. The basal forebrain cholinergic system is dependent on NGF and cholinergic basal forebrain neurons are the major cell group that expresses the receptor for NGF, i.e. TrkA. Although the role of NGF in cholinergic neuronal survival and function is well established, studies have also shown neuroprotective/neurorestorative effects mediated by this system, e.g. that axotomized cholinergic projections in animals can be rescued by TrkA activation (Lucidi-Phillipi C A, Neuron., 1996, 16(3):653-663).

An early morphological change in the brain of AD-patients is a decreased hippocampal volume. BDNF/TrkB-stimulated signalling has previously been shown to be necessary for survival and morphogenesis of especially hippocampal neurons. Moreover, it is widely accepted that BDNF plays a critical role in neuronal plasticity and long-term potentiation (LTP). Indeed, a growing body of experimental evidence suggests that increased BDNF signalling could potentially improve cognition in AD. The transplantation of stem cells into the brain of a triple-transgenic mouse model of AD, that expresses amyloid and tau pathology, i.e. the major neuropathological hallmarks of AD, results in improved cognition (Blurton-Jones M, PNAS, 2009. 106(32): p. 13594-13599). This effect is mediated by BDNF as gain-of-function studies show that recombinant BDNF mimics the beneficial effects of neural stem cell (NSC) transplantation. Furthermore, loss-of-function studies show that depletion of NSC-derived BDNF fails to improve cognition or restore hippocampal synaptic density.

Given the potent neuroprotective and neurorestorative effects of the TrkA/NGF and TrkB/BDNF systems, small molecule positive modulators of neurotrophin signalling might be beneficial in treating a number of diseases with neurodegeneration including, but not limited to, Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders. The modulators can also be used in the treatment of diseases where enhancement of nerve regeneration is beneficial, such as demyelinating diseases including, but not limited to, multiple sclerosis. The modulators could also be used for neuroprotection before or after an insult such as spinal cord injury, stroke, hypoxia, ischemia, brain injury including traumatic brain injury. Moreover, the important role of these neurotrophin systems in synaptic plasticity is thought to mediate learning and memory processes, and indicates that the modulators could also be used in disorders where cognitive function is impaired, including, but not limited to, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia.

Recent data have also indicated that NGF/TrkA and BDNF/TrkB systems may operate as metabotrophins, that is, be involved in the maintenance of cardiometabolic homeostasis (glucose and lipid metabolism as well as energy balance, cardioprotection, and wound healing) (Chaldakov G, Arch Ital Biol. 2011 Jun. 149(2):257-63). In fact, mutations in the genes encoding BDNF and its receptor TrkB have been shown to lead to severe obesity in humans (Yeo, G S. et al. Nat. Neurosci. 2004, 7, 1187-1189). Therefore, indications such as atherosclerosis, obesity, diabetes and metabolic syndrome could also benefit from NGF/TrkA and BDNF/TrkB directed therapies.

Another area of interest when it comes to neurotrophin signalling is neuropsychiatric disorders (Casten E et al., Neurobiol Dis. 2016 Jul 15, 30169-3). Studies have, for example, clearly demonstrated that depressed patients have reduced serum BDNF levels, which are restored after successful recovery (Shimizu et al., 2003, Sen et al., 2008). Moreover, several studies have demonstrated that chronic treatment with various antidepressant drugs increase BDNF mRNA and protein levels in the cerebral cortex and hippocampus (Calabrese et al., Psychopharmacology, 2011, 215, pp. 267-275). Also, local administration of BDNF into the brain has been shown to reduce depression-like behavior and mimic the effects of antidepressants (Hoshaw et al., Brain Res., 2005, 1037, pp. 204-208). Notably, the role for BDNF does not seem to be restricted to depression; it has also been implicated in other disorders, such as anxiety and schizophrenia (Castrén E., Handb. Exp. Pharmacol., 2014, 220, pp. 461-479). These data suggest that therapies targeting neurotrophin systems e.g. NGF/TrkA and BDNF/TrkB could have a therapeutic effect in several neuropsychiatric disorders, including, but not limited to, depression, schizophrenia and anxiety.

The finding that NGF and BDNF play important roles in neuronal homeostasis in combination with their neuroprotective and neurorestorative effect makes these pathways highly suitable as candidates for drug intervention for the treatment of diseases of the central nervous system and the peripheral nervous system. However, BDNF and NGF are themselves not ideal drug candidates due to their pharmacokinetic properties, the difficulties in administration and their limited ability to cross the blood-brain barrier. This has led to several attempts to identify peptides, cyclized peptides, peptide mimetics, small molecule agonist or selective modulators of NGF or BDNF. Several natural products such as gambogic amide (and analogues thereof), deoxygedunin and 7,8-dihydroxyflavone have been demonstrated to act as TrkA or TrkB agonists. Moreover, the tricyclic depressant amitriptyline has also been shown to be a TrkA and TrkB agonist. However, there is currently no specific TrkA or TrkB agonist that has reached the market. Therefore, there is an unmet need in the art for small molecule compounds that have the ability to stimulate or modulate TrkA and/or TrkB receptors, in combination with TrkC, FGFR1 and/or IGF1R and optionally other receptor tyrosine kinases for the treatment of both neurological and non-neurological disorders. There is still a need for compounds that have an improved potency and improved selectivity to TrkA and/or TrkB receptor.

BDNF production can be affected by a polymorphism within the BDNF gene (rs6265) causes a valine (Val) to methionine (Met) substitution at codon 66 (Val66Met). This polymorphism is found in approximately 30% of Caucasians and up to 70% in Asian populations. The presence of one or two Met alleles is associated with lower BDNF production in a subject. This lower BDNF production can lead to increased cognitive decline and decreased hippocampal volume.

A study by Boots et al (Neurology, 2017, 88, 1-9) demonstrated that subjects suffering sporadic Alzheimer's disease who carry the BDNF Met allele experience a steeper decline in episodic memory and executive function than non-carriers. Greater memory decline and decreased hippocampal function have also been observed in Val66Met patients with familial Alzheimer's disease (Lim et al., Brain, 2016, 139(10), 2766-2777).

The same study also showed increased tau-protein and phosphorylated tau-protein in the cerebrospinal fluid in this patient group. The decline in memory in subjects with pre-clinical or clinical Alzheimer's disease was exacerbated by greater amyloid plaque burden, thus suggesting that it is possible to treat Alzheimer's disease at various stages of the disease by potentiating the effects of BDNF in patients with the Val66Met polymorphism. Such treatment may lead to neuroprotection and increased cognitive function.

Toltrazuril (1-methyl-3-(3-methyl-4-{4-[(trifluoromethyl)sulfanyl]phenoxy}phenyl)-1,3,5-triazinane-2,4,6-trione; Baycox®) and its oxidised metabolites, particularly toltrazuril sulfone (ponazuril; Marquis®), are triazine-based antiprotozoal compounds that are used in veterinary medicine to treat coccidial infections, such as isosporiasis, toxoplasmosis, neosporosis, and equine protozoal meningoencephalitis.

A recent study by Suzuki et al. (FEBS Open Bio 2016, 6 461-468), reported that toltrazuril inhibits the binding of β-amyloid oligomers to ephrin type-B receptor 2 (EphB2; a receptor understood to play a role memory and learning functions) by 30%. However, due to a lack of similar inhibitory activity at the cellular prion protein (PrP^{C}), it was not selected for further studies as a potential candidate compound for the treatment of Alzheimer's disease.

The listing or discussion of an apparently prior-published document in this specification should not necessarily be taken as an acknowledgement that the document is part of the state of the art or is common general knowledge.

### SUMMARY OF THE INVENTION

It has now surprisingly been found that certain 4-phenoxy-phenyl-1,3,5-triazine derivatives, such as toltrazuril and oxidised derivatives thereof are positive modulators of Trk receptors (including TrkA, TrkB and TrkC) and receptor tyrosine kinases such as IGF1R and/or FGFR1, and thus have properties rendering them useful for the treatment of diseases characterised by impaired signalling of neurotrophins and/or other trophic factors, such as Alzheimer's disease. As a result of their mode of action, the compounds are unexpectedly particularly suitable as therapeutics for disorders such as Alzheimer's disease in patients having the the Val66Met mutation in the brain-derived neurotrophic factor (BDNF) gene.

This summary lists several embodiments of the presently disclosed subject matter, and in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In other words, for the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Disclosed herein are compounds having positive modulatory effect, either directly or indirectly, on the signalling mediated by the TrkA, TrkB and TrkC receptors, optionally in combination with a modulatory effect, either directly or indirectly, on the signalling mediated by receptor tyrosine receptors such as IGF1R and/or FGFR1 receptor molecule.

### Compounds for New Medical Uses

In a first aspect of the invention, there is provided a compound of formula I,

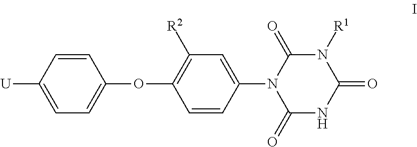

wherein:

R¹ represents phenyl optionally substituted by one or more (e.g. one) groups selected from C₁₋₄alkyl, —OC₁₋₄alkyl, halogen, —OC₁₋₄haloalkyl or methylenedioxy, thiophenyl optionally substituted by one or more (e.g. one) methyl groups, benzofuranyl, indolyl or, particularly, C₁₋₄alkyl,

R² represents OC₁₋₄alkyl optionally substituted by one or more (e.g. one) methoxy groups or, particularly, C₁₋₄alkyl and

U is selected from the group consisting of C₁₋₄haloalkyl-S—, C₁₋₄haloalkyl-S(O)— and C₁₋₄haloalkyl-S(O)₂—,

or a pharmaceutically-acceptable salt or prodrug thereof, for use in the treatment and/or prevention of a disease characterised by impaired signalling of neurotrophins and/or other trophic factors, in a patient with the Val66Met mutation in the brain-derived neurotrophic factor gene.

For the avoidance of doubt, compounds of formula I as defined herein, and pharmaceutically-acceptable salts thereof, may be referred to as “the compounds of the invention”. Further, for the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

In an alternative first aspect of the invention, there is provided a method of treating a disease characterised by impaired signalling of neurotrophins and/or other trophic factors, in a patient with the Val66Met mutation in the brain-derived neurotrophic factor gene comprising administering to a patient in need thereof a therapeutically effective amount of a compound of formula I, or a pharmaceutically acceptable salt or prodrug thereof, as hereinbefore defined.

In a further alternative first aspect of the invention, there is provided the use of a compound of formula I, or a pharmaceutically-acceptable salt or prodrug thereof, as hereinbefore defined, for the manufacture of a medicament for the treatment of a disease characterised by impaired signalling of neurotrophins and/or other trophic factors, in a patient with the Val66Met mutation in the brain-derived neurotrophic factor gene.

In particular embodiments (i.e. particular embodiments of the first aspects of the invention), the compound of formula I is such that R¹ represents phenyl optionally substituted by one or more (e.g. one) groups selected from C₁₋₂alkyl (e.g. methyl), —OC₁₋₂alkyl (e.g. methoxy), Cl, F, —OC₁₋₂haloalkyl (e.g. —OCF₃) or methylenedioxy, thiophenyl optionally substituted by one or more (e.g. one) methyl groups, benzofuranyl, indolyl or, particularly, C₁₋₄alkyl (e.g. methyl).

In further particular embodiments, R¹ represents phenyl optionally substituted by one group selected from methyl, —OCH₃, Cl, F, —OCF₃ or methylenedioxy, or, particularly, C₁₋₄alkyl (e.g. methyl).

In further particular embodiments, R² represents C₁₋₂alkyl, or OC₁₋₃alkyl optionally substituted by one or more (e.g. one) methoxy groups.

In further particular embodiments, R² represents methyl, methoxy (—OMe), ethoxy (—OEt), iso-propoxy (—OⁱPr) or —OCH₂CH₂OCH₃ (e.g. methyl).

In further particular embodiments, R¹ represents methyl.

In further particular embodiments R² represents methyl.

In further particular embodiments, U is selected from the group consisting of

C₁₋₂haloalkyl-S—, C₁₋₂haloalkyl-S(O)— and C₁₋₂haloalkyl-S(O)₂—.

In further particular embodiments, U is selected from the group consisting of CF₃S—, CF₃S(O)— and CF₃S(O)₂—.

In yet further particular embodiments

R¹ represents phenyl (optionally substituted as hereinbefore defined, or, preferably unsubstituted) or, particularly, C₁₋₄alkyl (e.g. C₁₋₂alkyl),

R² is C₁₋₄alkyl,

U is selected from the group consisting of C₁₋₄haloalkyl-S—, C₁₋₄haloalkyl-S(O)— and C₁₋₄haloalkyl-S(O)₂—

In yet further particular embodiments,

R¹ represents methyl or phenyl (e.g. methyl),

R² represents C₁₋₂alkyl (e.g. methyl),

U is selected from the group consisting of C₁₋₂fluoroalkyl-S— (e.g. CF₃S—), C₁₋₂fluoroalkyl-S(O)— (e.g. CF₃S(O)—) and C₁₋₂fluoroalkyl-S(O)₂— (e.g. CF₃S(O)₂).

In other particular embodiments, R¹ represents phenyl (i.e. unsubstituted phenyl).

A particular compound for use in accordance with the first aspect of the invention is 1-methyl-3-(3-methyl-4-{4-[(trifluoromethyl)sulfanyl]phenoxy}phenyl)-1,3,5-triazinane-2,4,6-trione (toltrazuril, COMPOUND 1), or a pharmaceutically acceptable salt or prodrug thereof.

A further compound for use in accordance with the first aspect of the invention is 1-methyl-3-[3-methyl-4-(4-trifluoromethanesulfonylphenoxy)phenyl]-1,3,5-triazinane-2,4,6-trione (COMPOUND 2), or a pharmaceutically acceptable salt or prodrug thereof.

A further compound for use in accordance with the first aspect of the invention is 1-methyl-3-[3-methyl-4-(4-trifluoromethanesulfinylphenoxy)phenyl]-1 ,3,5-triazinane-2,4,6-trione (COMPOUND 3), or a pharmaceutically acceptable salt or prodrug thereof.

It has also been found that the oxidised variants of toltrazuril (i.e. toltrazuril sulfone and toltrazuril sulfoxide) and analogues thereof are unexpectedly more effective positive modulators of TrK receptors than is toltrazuril. In diseases characterised by impaired signalling of neurotrophins and/or other trophic factors, such as Alzheimer's disease, the levels of neurotrophins can be reduced and thus, it is of utmost importance of the compounds to be able to stimulate the effects of the neurotrophins even at low NGF/BDNF-concentrations. Accordingly, the direct administration of these compounds has the potential to provide particularly effective treatments for diseases characterised by impaired signalling of neurotrophins and/or other trophic factors.

Thus, in a second aspect of the invention, there is provided a compound of formula I, as defined hereinbefore,

wherein:

R¹ and R² as defined hereinbefore in respect of (the various embodiments of) the first aspect of the invention, and

U is selected from the group consisting of C₁₋₄haloalkyl-S(O)— and C₁₋₄haloalkyl-S(O)₂—, or a pharmaceutically-acceptable salt or prodrug thereof, for use in the treatment of a disease characterised by impaired signalling of neurotrophins and/or other trophic factors.

In an alternative second aspect of the invention, there is provided a method of treating and/or preventing a disease characterised by impaired signalling of neurtrophins or other trophic factors, comprising administering to a patient in need thereof a therapeutically effective amount of a compound of formula I, or a pharmaceutically acceptable salt or prodrug thereof, as defined in respect of the second aspect of the invention.

In a further alternative second aspect of the invention, there is provided the use of a compound of formula I, or a pharmaceutically acceptable salt or prodrug thereof, as defined in respect of the second aspect of the invention, for the manufacture of a medicament for the treatment and/or prevention of a disease characterised by impaired signalling of neurotrophins and/or other trophic factors.

In particular embodiments (i.e. particular embodiments of the second aspect of the invention), the compound of formula I is such that

R¹ represents C₁₋₄alkyl

R² represents C₁₋₄alkyl,

U is selected from the group consisting of C₁₋₄haloalkyl-S(O)— and C₁₋₄haloalkyl-S(O)₂—,

In more particular embodiments

R¹ represents phenyl (i.e. unsubstituted phenyl) or, particularly, methyl,

R² represents C₁₋₂alkyl (e.g. Me),

U is selected from the group consisting of C₁₋₂fluoroalkyl-S(O)— (e.g. CF₃S(O)—) and

C₁₋₂fluoroalkyl-S(O)₂— (e.g. CF₃S(O)₂—).

In further particular embodiments of the second aspect of the invention,

R¹ represents methyl,

R² represents C₁₋₄alkyl,

U is selected from the group consisting of C₁₋₂fluoroalkyl-S(O)— and C₁₋₂fluoroalkyl-S(O)₂—.

In other particular embodiments of the second aspect of the invention, R¹ represents phenyl.

In further particular embodiments of the second aspect of the invention, U is selected from the group consisting of CF₃S(O)— and (e.g. CF₃S(O)₂—).

A particular compound for use in accordance with the second aspect of the invention is 1-methyl-3-[3-methyl-4-(4-trifluoromethanesulfonylphenoxy)phenyl]-1,3,5-triazinane-2,4,6-trione (COMPOUND 2), or a pharmaceutically acceptable salt or prodrug thereof.

A further particular compound for use in accordance with the second aspect of the invention is 1-methyl-3-[3-methyl-4-(4-trifluoromethanesulfinylphenoxy)phenyl]-1,3,5-triazinane-2,4,6-trione (COMPOUND 3), or a pharmaceutically acceptable salt or prodrug thereof.

In other particular embodiments of the first and second aspects of the invention, the compounds of formula I and pharmaceutically acceptable salts thereof are indicated for use as modulators of neurotrophin receptors, such as TrkA, TrkB, TrkC and/or their signalling and receptor tyrosine kinases, such as IGF1R and FGFR1 and/or their signalling in the treatment and/or prevention of both non-neurological and neurological diseases.

In particular instances, formula I is defined as

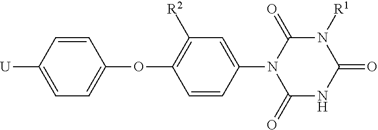

wherein:

R¹ represents C₁₋₄alkyl,

R² represents C₁₋₄alkyl, and

U is selected from the group comprising C₁₋₄haloalkyl-S—, C₁₋₄haloalkyl-S(O)— and C₁₋₄haloalkyl-S(O)₂—.

Further particular embodiments relate to the compound according to formula I, wherein

R¹ represents methyl,

R² represents C₁₋₂alkyl, and

U is selected from the group comprising C₁₋₂fluoroalkyl-S—, C₁₋₂fluoroalkyl-S(O)— and C₁₋₂fluoroalkyl-S(O)₂—.

Yet further particular embodiments relate to the compound according to formula I, wherein R¹ is methyl, R² is methyl, and U is selected from the group comprising trifluoromethylthio, trifluoromethylsulphonyl and trifluoromethylsulphinyl.

Another embodiment relates to the compound 1-methyl-3-(3-methyl-4-{4-[(trifluoromethyl)sulfanyl]phenoxy}phenyl)-1,3,5-triazinane-2,4,6-trione (COMPOUND 1) or pharmaceutical acceptable salt thereof, for use as positive modulators of neurotrophin receptors, such as TrkA, TrkB, TrkC and/or their signalling and receptor tyrosine kinases, such as FGFR1 and IGF1R and/or their signalling in the treatment and/or prevention of both non-neurological and neurological diseases.

A further embodiment relates to the compound 1-methyl-3-[3-methyl-4-(4-trifluoromethanesulfonylphenoxy)phenyl]-1,3,5-triazinane-2,4,6-trione (COMPOUND 2) or pharmaceutical acceptable salt thereof, for use as positive modulators of neurotrophin receptors, such as TrkA, TrkB, TrkC and/or their signalling and receptor tyrosine kinases, such as FGFR1 and IGF1R and/or their signalling in the treatment and/or prevention of both non-neurological and neurological diseases.

One embodiment relates to the compound 1-methyl-3-[3-methyl-4-(4-trifluoromethanesulfinylphenoxy)phenyl]-1,3,5-triazinane-2,4,6-trione (COMPOUND 3) or pharmaceutical acceptable salt thereof, for use as positive modulators of neurotrophin receptors, such as TrkA, TrkB, TrkC and/or their signalling and receptor tyrosine kinases, such as FGFR1 and IGF1R and/or their signalling in the treatment and/or prevention of both non-neurological and neurological diseases.

Certain compounds of the first and second aspects of the invention are novel and/or not previously disclosed for use in medicine.

Accordingly, in a third aspect of the invention, there is provided a compound of formula I, as defined hereinbefore

wherein:

R¹ represents C₂₋₄alkyl, phenyl optionally substituted by one or more groups selected from C₁₋₄alkyl, —OC₁₋₄alkyl, halogen, —OC₁₋₄haloalkyl or methylenedioxy, thiophenyl optionally substituted by one or more methyl groups, benzofuranyl or indolyl;

R² represents OC₁₋₄alkyl optionally substituted by one or more (e.g. one) methoxy groups or, particularly, C₁₋₄alkyl; and

U is selected from the group consisting of C₁₋₄haloalkyl-S—, C₁₋₄haloalkyl-S(O)— and C₁₋₄haloalkyl-S(O)₂—

or a pharmaceutically acceptable salt or prodrug thereof.

In particular embodiments, (i.e. particular embodiments of the third aspect of the invention), R¹ represents C₂₋₄alkyl, phenyl optionally substituted by one or more (e.g. one) groups selected from C₁₋₂alkyl (e.g. methyl), OC₁₋₂alkyl (e.g. —OCH₃), Cl, F, OC₁₋₂haloalkyl (e.g. —OCF₃) or methylenedioxy, thiophenyl optionally substituted by one or more (e.g. one) methyl groups, benzofuranyl or indolyl.

In further particular embodiments, R¹ represents phenyl optionally substituted by one or more (e.g. one) groups selected from C₁₋₂alkyl (e.g. methyl), —OC₁₋₂alkyl (e.g. —OCH₃), Cl, F, —OC₁₋₂haloalkyl (e.g. —OCF₃) or methylenedioxy, thiophenyl optionally substituted by one or more (e.g. one) methyl groups, benzofuranyl or indolyl.

In further particular embodiments, R¹ represents phenyl optionally substituted by one group selected from methyl, —OCH₃, Cl, F, —OCF₃ or methylenedioxy.

In further particular embodiments, R¹ represents phenyl.

In further particular embodiments, R² represents C₁₋₂alkyl or OC₁₋₃alkyl optionally substituted by one or more (e.g. one) OMe.

In further particular embodiments, R² represents methyl, methoxy, ethoxy, iso-propoxy or —OCH₂CH₂OCH₃ (e.g. methyl).

In further particular embodiments, U is selected from the group consisting of CF₃S—, CF₃S(O)— and CF₃S(O)₂—.

In further particular embodiments, the compound of formula I is such that:

R¹ represents C₂₋₄alkyl or phenyl (i.e. unsubstituted phenyl);

R² represents C₁₋₂alkyl (e.g. methyl);

U is selected from the group consisting of C₁₋₂fluoroalkyl-S—, C₁₋₂fluoroalkyl-S(O)— and C₁₋₂fluoroalkyl-S(O)₂—.

In more particular embodiments of the third aspect of the invention,

R¹ represents phenyl (i.e. unsubstituted phenyl);

R² represents methyl;

U is selected from the group consisting of CF₃S—, CF₃S(O)— and CF₃S(O)₂—.

Particular compounds of the third aspect of the invention are:

1-[-3-methyl-4-(4-trifluoromethanesulfinylphenoxy)phenyl]-3-phenyl-1,3,5-triazinane-2,4,6-trione (COMPOUND 4), and pharmaceutically acceptable salts or prodrugs thereof;

1-(-3-methyl-4-{4-[(trifluoromethyl)sulfanyl]phenoxy}phenyl)-3-phenyl-1,3,5-triazinane-2,4,6-trione (COMPOUND 5), and pharmaceutically acceptable salts or prodrugs thereof;

1-[-3-methyl-4-(4-trifluoromethanesulfonylphenoxy)phenyl]-3-phenyl-1,3,5-triazinane-2,4,6-trione (COMPOUND 6), and pharmaceutically acceptable salts or prodrugs thereof.

In a fourth aspect of the invention, there is provided the compounds of formula I as defined in (the various embodiments of) the second and third aspects of the invention, or pharmaceutically acceptable salt or prodrug thereof, for use in medicine.

In particular, there is provided a compound of formula I, wherein

R¹ represents phenyl or, particularly, C₁₋₄alkyl (e.g. methyl)

R² represents C₁₋₄alkyl (e.g. methyl); and

U is selected from the group consisting of C₁₋₄haloalkyl-S(O)— (e.g. CF₃S(O)—) and C₁₋₄haloalkyl-S(O)₂— (e.g. CF₃S(O)₂—),

or a pharmaceutically-acceptable salt thereof, for use in medicine.

In a further embodiment, there is provided a compound of formula I, wherein

R¹ represents C₂₋₄alkyl or, particularly, phenyl;

R² represents C₁₋₄alkyl (e.g. methyl); and

U is selected from the group consisting of C₁₋₄haloalkyl-S— (e.g. CF₃S—), S(O)— (e.g. CF₃S(O)—) and C₁₋₄haloalkyl-S(O)₂— (e.g. CF₃S(O)₂—),

or a pharmaceutically acceptable salt thereof, for use in medicine.

In a yet further embodiment, there is provided a a compound of formula I, wherein

R¹ represents C₂₋₄alkyl or, particularly, phenyl;

R² represents C₁₋₄alkyl; and

U is selected from the group consisting of C₁₋₄haloalkyl-S— (e.g. CF₃S—), C₁₋₄haloalkyl-S(O)— (e.g. CF₃S(O)—) and C₁₋₄haloalkyl-S(O)₂— (e.g. CF₃S(O)₂—),

or a pharmaceutically acceptable salt thereof, for use in the treatment of a disease characterised by impaired signalling of neurotrophins and/or other trophic factors, particularly in a patient with the Val66Met mutation in the BDNF gene.

As used herein, the phrase “for use in medicine” will be understood to refer to use in human medicine only, and does not include veterinary use.

### Medical Uses

As described hereinbefore, the compounds of the first to fourth aspects of the invention are useful in the treatment of diseases characterised by impaired signalling of neurotrophins and/or other trophic factors. Due to their mode of action, the compounds have particular utility in the treatment of such diseases in patients with the Val66Met mutation in the BDNF gene.

The skilled person will understand that trophic factors refer to a class of molecules that promote the growth and maintenance of cellular tissues. Neurotrophins may be understood to refer to a class of molecules associated with promoting the growth and survival of neurons, which are also referred to as neurotrophic factors. Examples of neurotrophins include NGF, BDNF, NT3 and NT4/5. Other trophic factors include insulin-like growth factor (IGF-1), fibroblast growth factors (FGFs), hepatocyte growth factor (HGF) and glial cell line-derived neurotrophic factors such as glial cell-derived neurotrophic factor (GDNF), Neurturin (NRTN), artemin (ARTN) and persephin (PSPN).

As used herein, the phrase diseases characterised by impaired signalling of neurotrophins and other trophic factors may be understood to indicate diseases and disorders that involve reduced signalling of trophic factors, such as those listed above. Such disorders may be treated through the positive modulation of neurotrophin receptors, such as TrKA, TrKB and TrkC and/or their signalling, and receptor tyrosine kinases such as FGFR1 and IGF1R and/or their signalling and/or the positive modulation of other trophic factor receptors.

The Val66Met mutation in the BDNF gene refers to a common single-nucleotide polymorphism in the brain-derived neurotrophic factor (BDNF) gene, resulting in a methionine (Met) substitution for valine (Val) at codon 66 (Val66Met).

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of Alzheimer's disease, the term may refer to achieving an improvement in cognition in the patient being treated.

As used herein, the term prevention (and, similarly, preventing) will include references to the prophylaxis of the disease or disorder (and vice-versa). As such, references to prevention may also be references to prophylaxis, and vice versa. In particular, such terms may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to a patient (or to patients) will refer to a living subject being treated, including mammalian (e.g. human) patients. In particular, references to a patient will refer to human patients.

For the avoidance of doubt, the skilled person will understand that such treatment or prevention will be performed in a patient (or subject) in need thereof. The need of a patient (or subject) for such treatment or prevention may be assessed by those skilled the art using routine techniques.

As used herein, the terms disease and disorder (and, similarly, the terms condition, illness, medical problem, and the like) may be used interchangeably.

The 4-phenoxy-phenyl-1,3,5-triazine derivatives are modulators of neurotrophin receptors, such as TrkA, TrkB, TrkC and/or their signalling and receptor tyrosine kinases, such as FGFR1 and IGF1R and/or their signalling. The compounds are believed to have an improved potency for the modulation of neurotrophin receptors, such as TrkA, TrkB, TrkC and/or their signalling and receptor tyrosine kinases, such as FGFR1 and IGF1R and/or their signalling. It is believed that the compounds of the invention would have a reduced potential for side effects associated with conventional agonists for TrkA and TrkB.

Another indication includes setting in which there is a goal for enhancing plasticity of the nervous system, such as during rehabilitation or acquisition of a new learned physical or intellectual skill. Moreover, it also includes facilitation of neuronal or non-neuronal or stem cell survival or promoting neural function by treating a neural or non-neuronal or stem cell with a compound of the invention having the ability to have a positive modulatory effect, either directly or indirectly, on the signalling mediated by the TrkA, TrkB lo and TrkC receptors, optionally in combination with a modulatory effect, either directly or indirectly, on on the signalling mediated by receptor tyrosine kinases such as IGF1R and/or FGFR1 receptor.

The invention relates to the compound of formula I, or a pharmaceutically acceptable salt thereof, as defined above, for use in therapy. Without being bound to theory regarding the mode of action of the compounds defined above, it is believed that the compounds can be used for treatment and/or prevention of the diseases mentioned below.

In particular embodiments, the diseases that may be treated by compounds of formula I include Alzheimer's disease, depression, Parkinson's disease, other Parkinsonian disorders and/or other tauopathies, Lewy body dementia, multiple sclerosis, Huntington's disease, mild cognitive impairment, brain injuries (including traumatic brain injuries), stroke, other dementia disorders, motorneurone diseases, Pick disease, spinal chord injury, hypoxic ischemia injury, cognitive dysfunction, coronary artery disease, obesity, metabolic syndrome, diabetes, Charcot-Marie-Tooth disease, diabetic neuropathy, tissue regeneration, motor function, nerve injury, hearing loss, blindness, posterior eye diseases, dry eye disease, neurotrophic keratitis, glaucoma, high intraocular pressure (IOP), retinitis pigmentosa, post-traumatic stress disorders, WAGR syndrome, diseases of the olfactory tract, olfactory decline, olfactory dysfunction, anxiety, fragile X syndrome, congenital central hypoventilation syndrome, obsessive-compulsive disorder, generalized anxiety disorder, eating disorders, bipolar disorder, chronic fatigue syndrome, neuromyelitis optica, Rett syndrome, Friedrich's ataxia and obstructive sleep apnea-hypopnea syndrome.

As used herein, the phrase “other Parkinsonian disorders” may be understood to refer to disorders that have symptoms similar to Parkinson's disease, such as bradykinesia, tremors and postural instability. Examples of such disorders include progressive supranuclear palsy (PSP), multiple system atrophy (MSA), and corticobasal degeneration (CBD).

The phrase “other tauopathies” may be understood to refer to neurodegenerative diseases other than Alzheimer's disease that are associated with the pathological misfolding of tau protein in the brain. Examples of such disorders include primary age-related tauopathy, progressive supranuclear palsy, Pick's disease, corticobasal degeneration and post-encephalitic parkinsonism. The skilled person will understand that certain disorders such as progressive supranuclear palsy may be described as both a Parkinsonian disorder and a tauopathy.

The phrase “other dementia disorders” may be understood to include vascular dementia, mixed vascular dementia, incident dementia, post-operative dementia, presenile dementia, dementia associated with Parkinson's disease and dementia due to HIV infection. Progressive supranuclear palsy and corticobasal degeneration may also be classed as dementia disorders.

Motorneurone diseases include amyotrophic lateral sclerosis (ALS), hereditary spastic paraplegia (HSP), primary lateral sclerosis (PLS), progressive muscular atrophy (PMA), progressive bulbar palsy (PBP) and pseudobulbar palsy.

Cognitive dysfunction may be understood to refer to reduced cognitive abilities in a patient including reduced ability in learning, memory loss, perception, and problem solving. Cognitive dysfunction is associated with a range of conditions, such as Alzheimer's disease, Parkinson's disease, progressive supranuclear palsy, corticobasal degeneration and schizophrenia. Accordingly, in particular embodiments, the compounds of the invention are for use in the treatment of cognitive dysfunction in Alzheimer's disease, Parkinson's disease, progressive supranuclear palsy, corticobasal degeneration or schizophrenia. Cognitive dysfunction also includes post-operative cognitive dysfunction and impaired cognition associated with preterm delivery.

Similarly, in other particular embodiments, the compounds of the invention are for use in improving cognition in a patient with Alzheimer's disease, Parkinson's disease, progressive supranuclear palsy, corticobasal degeneration or schizophrenia. As used herein, the phrase “improving cognition” may be understood to indicate enhancing a patient's learning, memory, perception, and/or problem-solving ability. Improving cognition may also refer to slowing or arresting the rate of decline in cognition in a patient suffering from cognitive dysfunction (e.g. associated with the disorders listed above).

Cognitive function may be assessed using standard tests known to the person skilled in the art. Examples of such tests include the Alzheimer's Disease Assessment Scale-Cognitive subscale test (ADAS-COG) the Mini-Mental State Examination (MMSE), the Clinical Dementia Rating (CDR) the Clinical Dementia Rating-Sum of Boxes (CDR-SB), the Alzheimer's Disease Cooperative Study—Preclinical Alzheimer Cognitive Composite (ADCS-PACC) and the Repeatable Battery for the Assessment of Neuropsychological Status (RBANS) test.

As used herein, “eating disorders” may be understood to include hyperphagia, anorexia nervosa, restricting anorexia nervosa and bulimia nervosa.

In other particular embodiments of the first and second aspects of the invention, there is provided the compounds of formula I, such as COMPOUND 1 or, particularly COMPOUND 2 or COMPOUND 3, as defined above, or any mixture thereof, or a pharmaceutically acceptable salt thereof, for use in treatment and/or prevention of one or more disease selected from the group comprising or containing Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, depression, obesity, metabolic syndrome, pain, depression, schizophrenia and anxiety.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motorneuron disease, Pick disease, obesity, metabolic syndrome, diabetes and Rett syndrome. The treatment of this group of disorders may be particularly effective in patients having the Val66Met mutation in the BDNF gene.

In yet more particular embodiments, the disease characterised by impaired signalling of neurotrophins and/or other trophic factors is selected from the group consisting of Alzheimer's disease, Parkinson's disease, Cognitive dysfunction, depression and Rett Syndrome.

An embodiment relates to the compound of formula I, COMPOUND 1 or, particularly, COMPOUND 2 or COMPOUND 3, as defined above, as defined above, or any mixture thereof, or a pharmaceutically acceptable salt thereof, for use in treatment and/or prevention of Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other Parkinsonian disorders.

Another embodiment relates to the compound of formula I, COMPOUND 1 or, particularly, COMPOUND 2 or COMPOUND 3, as defined above, as defined above, or any mixture thereof, or a pharmaceutically acceptable salt thereof, for use in treatment and/or prevention of Alzheimer's disease, Parkinson's disease, Cognitive dysfunction in Schizophrenia, Rett's Syndrome and/or depression.

A further embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention of Alzheimer's disease.

An embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention of depression.

One embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention of a disease where enhancement of nerve regeneration is beneficial, such as demyelinating diseases.

A further embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention of multiple sclerosis.

A further embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention of Rett syndrome.

Another embodiment relates to the compound of formula I, COMPOUND 1 or, particularly, COMPOUND 2 or COMPOUND 3, as defined above, or any mixture thereof, or a pharmaceutically acceptable salt thereof, for use in treatment and/or prevention spinal cord injury, stroke, hypoxia, ischemia and/or brain injury including traumatic brain injury.

In one embodiment, the invention relates to a compound of formula I, COMPOUND 1 or, particularly, COMPOUND 2 or COMPOUND 3, as defined above, or any mixture thereof, or a pharmaceutically acceptable salt thereof , for use in the treatment and/or prevention of mild cognitive impairment, dementia disorders (including dementia of mixed vascular and degenerative origin, presenile dementia, senile dementia and dementia associated with Parkinson's disease, progressive supranuclear palsy, corticobasal degeneration, post-operative dementia) and/or cognitive dysfunction in schizophrenia.

In a further embodiment, the invention relates to a compound of formula I, COMPOUND 1 or, particularly, COMPOUND 2 or COMPOUND 3, as defined above, or any mixture thereof, or a pharmaceutically acceptable salt thereof , for use in the treatment and/or prevention of atherosclerosis, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, depression, obesity, metabolic syndrome and/or pain

In yet a further embodiment, the invention relates to a compound of formula I, COMPOUND 1 or, particularly, COMPOUND 2 or COMPOUND 3, as defined above, or any mixture thereof, or a pharmaceutically acceptable salt thereof, for use in the treatment and/or prevention of depression, schizophrenia and/or anxiety.

Another embodiment relates to a use of the compound of formula I, COMPOUND 1 or, particularly, COMPOUND 2 or COMPOUND 3, as defined above, or any mixture thereof, or a pharmaceutically acceptable salt thereof, in the treatment and/or prevention of a disease in which modulators of neurotrophin receptors, such as TrkA, TrkB, TrkC and/or their signalling and receptor tyrosine kinases, such as FGFR1 and IGF1R and/or their signalling are beneficial, such as in the treatment and/or prevention of both non-neurological and neurological diseases.

A further embodiment relates to a use of the compound of formula I, COMPOUND 1 or, particularly, COMPOUND 2 or COMPOUND 3, or any mixture thereof, or a pharmaceutically acceptable salt thereof, as defined above, in the treatment and/or prevention of one or more disease selected from the group comprising or containing Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other Parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, atherosclerosis, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, depression, obesity, metabolic syndrome, pain and cancer, depression, schizophrenia and anxiety.

The invention relates to the use of a compound of the invention in a method of treating, preventing or reducing the risk of a disease in which modulators of neurotrophin receptors, such as TrkA, TrkB, TrkC and/or their signalling and receptor tyrosine kinases, such as FGFR1 and IGF1R and/or their signalling, are beneficial, such as in the treatment and/or prevention of both non-neurological and neurological diseases.

One embodiment relates to the use of a compound of the invention a method of treating, preventing or reducing the risk of, one or more disease selected from the group comprising or containing Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders, enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, atherosclerosis, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, diseases of the olfactory tract, depression, obesity, metabolic syndrome, pain and cancer, depression, schizophrenia and anxiety, which comprises administering to a mammal, such as a human, in need thereof, a therapeutically effective amount of a compound of formula I, COMPOUND 1 or, particularly, COMPOUND 2 or COMPOUND 3, as defined above, or any mixture thereof, or a pharmaceutically acceptable salt thereof.

Another embodiment relates to the use of a compound of the invention in said method of treating, preventing or reducing the risk of Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other parkinsonian disorders.

An embodiment relates to the use of a compound of the invention in said method of treating, preventing or reducing the risk of Alzheimer's disease, Parkinson's disease, Cognitive dysfunction in Schizophrenia, Rett's Syndrome and/or Depression.

A further embodiment relates to the use of a compound of the invention in said method of treating, preventing or reducing the risk of a disease where enhancement of nerve regeneration is beneficial such as demyelinating diseases, such as multiple sclerosis.

Yet a further embodiment relates to the use of a compound of the invention in said method of treating, preventing or reducing the risk of spinal cord injury, stroke, hypoxia, ischemia and/or brain injury including traumatic brain injury.

Another embodiment relates to the use of a compound of the invention in said method of treating, preventing or reducing the risk of mild cognitive impairment, dementia disorders (including dementia of mixed vascular and degenerative origin, presenile dementia, senile dementia and dementia associated with Parkinson's disease, progressive supranuclear palsy or corticobasal degeneration) and/or cognitive dysfunction in lo schizophrenia.

One embodiment relates to the use of a compound of the invention in said method of treating, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, depression, obesity, metabolic syndrome and/or pain

Yet another embodiment relates to the use of a compound of the invention in said method of treating, preventing or reducing the risk of depression, schizophrenia and/or anxiety.

A particular embodiment to be mentioned is

1-methyl-3-[3-methyl-4-(4-trifluoromethanesulfonylphenoxy)phenyl]-1,3,5-triazinane-2,4,6-trione (COMPOUND 2), or a pharmaceutically acceptable salt thereof, for use in the treatment of Alzheimer's disease.

A further particular embodiment to be mentioned is

1-methyl-3-[3-methyl-4-(4-trifluoromethanesulfinylphenoxy)phenyl]-1,3,5-triazinane-2,4,6-trione (COMPOUND 3), or a pharmaceutically acceptable salt thereof, for use in the treatment of Alzheimer's disease.

A further particular embodiment to be mentioned is

1-methyl-3-(3-methyl-4-{4-[(trifluoromethyl)sulfanyl]phenoxy}phenyl)-1,3,5-triazinane-2,4,6-trione (COMPOUND 1) or a pharmaceutically acceptable salt thereof, for use in the treatment of Alzheimer's disease in a patient with the Val66Met mutation in the BDNF gene.

A further particular embodiment to be mentioned is

1-methyl-3-[3-methyl-4-(4-trifluoromethanesulfonylphenoxy)phenyl]-1,3,5-triazinane-2,4,6-trione (COMPOUND 2), or a pharmaceutically acceptable salt thereof, for use in the treatment of Alzheimer's disease in a patient with the Val66Met mutation in the BDNF gene.

A further particular embodiment to be mentioned is

1-methyl-3-[3-methyl-4-(4-trifluoromethanesulfinylphenoxy)phenyl]-1,3,5-triazinane-2,4,6-trione (COMPOUND 3), or a pharmaceutically acceptable salt thereof, for use in the treatment of Alzheimer's disease in a patient with the Val66Met mutation in the BDNF gene.

### Pharmaceutical Compositions

As described herein, compounds as defined for any of the first to third aspects of the invention are useful as pharmaceuticals. Such compounds may be administered alone or may be administered by way of known pharmaceutical compositions/formulations.

In a fifth aspect of the invention, there is provided a pharmaceutical composition comprising a compound of formula I as defined in (the various embodiments of) the second or third aspects of the invention, or pharmaceutically acceptable salt or prodrug thereof, and optionally a pharmaceutically acceptable adjuvant, diluent or carrier.

In a sixth aspect of the invention, there is provided a pharmaceutical composition comprising a compound as defined in any one of (the various embodiments of) the first to third aspects of the invention, or a pharmaceutically-acceptable salt or prodrug thereof, and optionally a pharmaceutically acceptable adjuvant, diluent or carrier for use in the treatment of a disease characterised by impaired signalling of neurotrophins and/or other trophic factors (including the various diseases and disorders listed herein), optionally in a patient with the Val66Met mutation in the BDNF gene.

In a particular embodiment, there is provided a pharmaceutical composition comprising a compound as defined in any one of (the various embodiments of) the first aspect of the invention, or a pharmaceutically acceptable salt or prodrug thereof, and optionally a pharmaceutically acceptable adjuvant, diluent or carrier for use in the treatment of a disease characterised by impaired signalling of neurotrophins and/or other trophic factors (including the various diseases and disorders listed herein), in a patient with the Val66Met mutation in the BDNF gene.

In a further particular embodiment, there is provided a pharmaceutical composition comprising a compound as defined in any one of (the various embodiments of) the second or third aspect of the invention, or a pharmaceutically acceptable salt or prodrug thereof, and optionally a pharmaceutically acceptable adjuvant, diluent or carrier for use in the treatment of a disease characterised by impaired signalling of neurotrophins and/or other trophic factors (including the various diseases and disorders listed herein).

The skilled person will understand that compounds of the invention may act systemically and/or locally (i.e. at a particular site), and may therefore be administered accordingly using suitable techniques known to those skilled in the art.

The skilled person will understand that compounds and compositions as described herein will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form.

Conventional procedures for the selection and preparation of suitable pharmaceutical formulations are described in, for example, “Pharmaceuticals—The Science of Dosage Form Designs”, M. E. Aulton, Churchill Livingstone, 1988. For preparing pharmaceutical compositions from the compounds of the invention, inert, pharmaceutically acceptable carriers can be either solid or liquid. Solid form preparations include powders, tablets, dispersible granules, capsules, cachets, and suppositories.

Pharmaceutical compositions as described herein will include formulations in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration. In particular, compounds may be formulated for local delivery to the CNS, for example in the form of artificial cerebrospinal fluid (CSF).

Thus, in particular embodiments, the pharmaceutical composition is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

Thus, compounds, of the present invention, and compositions comprising the same, may be administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

For example, in the preparation of pharmaceutical compositions for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Other excipients that may be used in the liquid preparations include amino sugars such as meglumine and cyclodextrin derivatives. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Depending on the mode of administration, the pharmaceutical composition will preferably comprise from 0.05 to 99 % wt (per cent by weight), more preferably from 0.05 to 80 % wt, still more preferably from 0.10 to 70 % wt, and even more preferably from 0.10 to 50 % wt, of compounds of the invention all percentages by weight being based on total composition.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in uses or methods of the invention.

More, particularly, the skilled person will understand that compounds of the invention may be administered (for example, as formulations as described hereinbefore) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 25 mg, 50 mg, 100 mg or 200 mg twice daily).

The optimum dosage and frequency of administration will depend on the particular condition being treated and its severity; the age, sex, size and weight, diet, and general physical condition of the particular patient; other medication the patient may be taking; the route of administration; the formulation; and various other factors known to physicians and others skilled in the art.

For the avoidance of doubt, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. Although the above-mentioned dosages are exemplary of the average case, there can, of course, be individual instances where higher or lower dosage ranges are merited, and such doses are within the scope of the invention.

In particular embodiments, the invention further relates to a pharmaceutical composition comprising the compound of formula I, COMPOUND 1 or, particularly, COMPOUND 2 or COMPOUND 3, as defined above, or any mixture thereof, or a pharmaceutically acceptable salt thereof, in the association with a pharmaceutically acceptable adjuvant, dilutent or carrier.

The invention also relates to a process for the preparation of a pharmaceutical composition, as defined above, which comprises mixing a compound of formula I, COMPOUND 1 or, particularly, COMPOUND 2 or COMPOUND 3, as defined above, or any mixture thereof, or a pharmaceutically acceptable salt thereof, with a pharmaceutically acceptable adjuvant, diluent or carrier.

One embodiment relates to a use of the pharmaceutical composition, as defined above, in therapy, or for the treatment and/or prevention of a disease in which modulators of neurotrophin receptors, such as TrkA, TrkB, TrkC and/or their signalling and receptor tyrosine kinases, such as FGFR1 and IGF1R and/or their signalling, are beneficial, such as in the treatment and/or prevention of both non-neurological and neurological diseases. Examples of such disease may be selected from the group comprising or containing Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders, enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy post-operative dementia, or corticobasal degeneration) and cognitive dysfunction in schizophrenia, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, diseases of the olfactory tract depression, obesity, metabolic syndrome, pain and cancer, depression, schizophrenia and anxiety.

The invention also relates to the use of a compound of formula I, COMPOUND 1 or particularly, COMPOUND 2 or COMPOUND 3, as defined above, or any mixture thereof, or a pharmaceutically acceptable salt thereof, in the manufacture of a medicament for the treatment and/or prevention of a disease in which modulators of neurotrophin receptors, such as TrkA, TrkB, TrkC and/or their signalling and receptor tyrosine kinases, such as FGFR1 and IGF1R and/or their signalling, are beneficial, such as in the treatment and/or prevention of both non-neurological and neurological diseases. Examples of such disease may be selected from the group comprising or containing Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders, enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including Charcot Marie Tooth and its variants, nerve transplantation and its complications, motor neuron disease, peripheral nerve injury, genetic or acquired or traumatic hearing loss, blindness and posterior eye diseases, depression, obesity, metabolic syndrome, pain and cancer, schizophrenia and anxiety.

### Combinations and Kits-of-Parts

The treatment and/or prevention of diseases of the nervous system and related pathology defined herein may be applied as a sole therapy or may involve, in addition to the compound of the invention, conjoint treatment with conventional therapy of value in treating one or more disease conditions referred to herein. Such conventional therapy may include one or more agents such as acetyl cholinesterase inhibitors, anti-inflammatory agents, cognitive and/or memory enhancing agents, atypical antipsychotic agents, dopamine agonists and/or L-DOPA.

Such conjoint treatment and/or prevention may be achieved by way of the simultaneous, sequential or separate dosing of the individual compounds of the invention or additional agents of the treatment and/or prevention. Such combination products employ the compounds, or pharmaceutically acceptable salts thereof, of the invention.

Accordingly, the skilled person will understand that treatment with compounds of the invention may further comprise (i.e. be combined with) further treatment(s) or preventative methods for the same condition. In particular, treatment with compounds of the invention may be combined with means for the treatment of diseases characterised by impaired signalling of neurotrophins and/or other trophic factors (such as Alzheimer's disease, Parkinson's disease, cognitive dysfunction and depression as described herein, e.g. Alzheimer's disease) such as treatment with one or more other therapeutic agent that is useful in the in the treatment the various diseases characterised by impaired signalling of neurotrophins and/or other trophic factors described herein, and/or one or more physical method used in the treatment (such as treatment through surgery), as known to those skilled in the art.

As described herein, compounds of the invention may also be combined with one or more other (i.e. different) therapeutic agents (i.e. agents that are not compounds of the invention) that are useful in the treatment and/or prevention of diseases characterised by impaired signalling of neurotrophins and/or other trophic factors. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a seventh aspect of the invention, there is provided a combination product comprising:
(I) a compound as defined in accordance with the second and third aspects of the invention, or a pharmaceutically acceptable salt or prodrug thereof; and
(II) one or more other therapeutic agent that is useful in the treatment or prevention of a disease characterised by impaired signalling of neurotrophins and/or other trophic factors,

wherein each of components (I) and (II) is fomulated in admixture, optionally with a pharmaceutically acceptable adjuvant diluent or carrier.

According to an eighth aspect of the invention, there is provided a kit-of-parts comprising:
(a) a pharmaceutical composition as defined in accordance with the fifth aspect of the invention; and
(b) a pharmaceutical composition comprising one or more other therapeutic agent that is useful in the treatment or prevention of a disease characterised by impaired signalling of neurotrophins and/or other trophic factors, optionally in admixture with one or more pharmaceutically-acceptable excipient,

which components (a) and (b) are each provided in a form that is suitable for administration in conjunction with the other.

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients are administered (optionally repeatedly) either together, or sufficiently closely in time, to lo enable a beneficial effect for the patient, that is greater, over the course of the treatment and/or prevention of the relevant condition, than if either agent is administered (optionally repeatedly) alone, in the absence of the other component, over the same course of treatment and/or prevention. Determination of whether a combination provides a greater beneficial effect in respect of, and over the course of, treatment or prevention of a particular condition will depend upon the condition to be treated or prevented, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of a disease characterised by impaired signalling of neurotrophins and/or other trophic factors, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Other therapeutic agents useful in the treatment or prevention of diseases characterised by impaired signalling of neurotrophins and/or other trophic factors will be well-known to those skilled in the art. For example, such other therapeutic agents may include: acetyl cholinesterase inhibitors, anti-inflammatory agents, cognitive enhancing agents, memory enhancing agents, and atypical antipsychotic agents, anti-depressive agents, anti-Alzheimer agents, beta-secretase inhibitors, gamma-secretase modulators, agents modifying tau function, amyloid-beta production inhibitors, antibodies directed at amyloid-beta, antibodies directed at tau, antibodies directed at alpha-synuclein, anti-Parkinson agents, anti-diabetic agents, anti-multiple sclerosis agents, anti-obesity agents, agents used for treatment of auditory dysfunction, agents used for treatment of ocular disease, agents used for the treatment of olfactory dysfunction, agents used for the treatment of gustatory dysfunction, anti-huntington agents, anti-Rett syndrome agents, anti-stroke agents. Particular therapeutic agents that may be mentioned include acetyl cholinesterase inhibitors, anti-Alzheimer agents, anti-Parkinson agents, cognitive enhancing agents, antibodies directed at amyloid-beta, antibodies directed at tau, antibodies directed at alpha-synuclein, beta-secretase inhibitors, gamma-secretase modulators,

Particular embodiments of combination products that may be mentioned include: pharmaceutical compositions comprising (i) a compound of formula I, COMPOUND 1, COMPOUND 2 or COMPOUND 3 (for example COMPOUND 2 or COMPOUND 3), as defined above, or any mixture thereof, or a pharmaceutically acceptable salt thereof, (ii) an additional therapeutic agent, or a pharmaceutically acceptable salt thereof, and (iii) a pharmaceutically acceptable excipient, carrier or diluent.

Further particular embodiments relate to pharmaceutical compositions comprising (i) a compound of formula I, COMPOUND 1, COMPOUND 2 or COMPOUND 3 (for example COMPOUND 2 or COMPOUND 3), as defined above, or any mixture thereof, or a pharmaceutically acceptable salt thereof, (ii) at least one agent selected from the group consisting of acetyl cholinesterase inhibitors, anti-inflammatory agents, cognitive enhancing agents, memory enhancing agents, and atypical antipsychotic agents, anti-depressive agents, anti-Alzheimer agents, beta-secretase inhibitors, gamma-secretase modulators, agents modifying tau function, amyloid-beta production inhibitors, antibodies directed at amyloid-beta, antibodies directed at tau, antibodies directed at alpha-synuclein, anti-Parkinson agents, anti-diabetic agents, anti-mutiple sclerosis agents, anti-obesity agents, agents used for treatment of auditory dysfunction, agents used for treatment of ocular disease, agents used for the treatment of olfactory dysfunction, agents used for the treatment of gustatory dysfunction, anti-huntington agents, anti-Rett syndrome agents, anti-stroke agents and (iii) a pharmaceutically acceptable excipient, carrier or diluent.

### Preparation of Compounds/Compositions

Pharmaceutical compositions/formulations, combination products and kits as described herein may be prepared in accordance with standard and/or accepted pharmaceutical practice.

Thus, in a ninth of the invention there is provided a process for the preparation of a pharmaceutical composition/formulation, as hereinbefore defined, which process comprises bringing into association a compound of the invention, as hereinbefore defined, or a prodrug thereof, with one or more pharmaceutically-acceptable excipient.

In further aspects (e.g. tenth and eleventh) of the invention, there is provided a process for the preparation of a combination product or kit-of-parts as hereinbefore defined, which process comprises bringing into association a compound of the invention, as hereinbefore defined, or a prodrug thereof, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention can be prepared as a free base or a pharmaceutically acceptable salt thereof by processes that are well known to those skilled in the art, such as those described in various patents, and those described in the examples provided hereinafter. For example, in U.S. Pat. No. 4,219,552, which is hereby included by reference, especially examples 2, 3 on column 10 and example 15 on column 11 of U.S. Pat. No. 4,219,552.

According to a twelfth aspect of the invention there is provided a process for the preparation of a compound of the invention as hereinbefore defined, comprising the step of:

reacting a compound of formula II

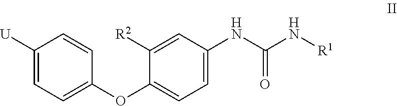

wherein R¹, R² and U are as defined hereinbefore, particularly as defined in accordance with the third aspect of the invention,

with a compound of formula III

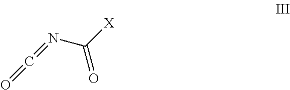

wherein X represents a suitable leaving group (e.g. —Oalkyl, —Cl), in the presence of a suitable solvent (for example apolar aprotic solvents such as toluene).

Alternatively, reacting a compound of formula II with with a suitable base, such as sodium hydride, at a temperature between 0° C. and room temperature for a suitable time (such as 1-60 minutes) in a suitable solvent such as DMF. This is followed by the addition of a compound of formula III to this mixture at a temperature between 0° C. and RT and stirred for a suitable time (such as 1-60 minutes).

Compounds of formulae II and III are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3rd edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula II may be prepared by reaction of a compound of formula IV

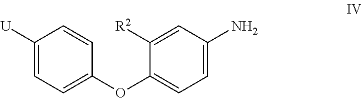

wherein R² and U are as defined hereinbefore, particularly as defined in accordance with the third aspect of the invention, with a compound of formula V

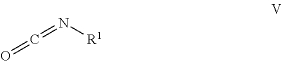

wherein R¹ is as defined hereinbefore, particularly as defined in accordance with the third aspect of the invention, in the presence of a suitable base (such as an organic amine base (e.g. triethylamine or N, N-diisopropylethylamine)) and a suitable solvent (such as dichloromethane).

Alternatively, a compound of formula IV may be reacted with a compound of formula VI

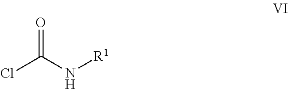

wherein R¹ is as defined hereinbefore, particularly in accordance with the third aspect of the invention, in the presence of a suitable base (e.g. triethylamine) and a suitable solvent (such as dichloromethane).

Similarly, compounds of formulae IV, V and VI are either commercially available, are known in the literature, or may be obtained either by analogy with the processes described herein, or by conventional synthetic procedures, in accordance with standard techniques, from available starting materials using appropriate reagents and reaction conditions.

The skilled person will understand that the substituents as defined herein, and substituents thereon, may be modified one or more times, after or during the processes described above for the preparation of compounds of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

Without wishing to be bound by theory, it is believed that the compounds of the invention provide novel therapies for the treatment of disorders characterised by impaired signalling of neurotrophins and/or other trophic factors, such as Alzheimer's disease. The ability of the compounds to modulate neurotrophin signalling through the modulation of receptors, such as TrkA, TrkB, TrkC and associated receptor tyrosine kinases, such as FGFR1 and IGF1R, indicates that they may be particularly suitable for the treatment of disorders in patients having the Val66Met mutation in the BDNF gene.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

Unless indicated otherwise, all technical and scientific terms used herein will have their common meaning as understood by one of ordinary skill in the art to which this invention pertains.

The definitions set forth in this application are intended to clarify terms used throughout this application. The term “herein” means the entire application.

As used herein, the term “disease” is intended to include disorder, condition or any equivalent thereof.

As used herein, the term “dementia” is intended to include a disease that describes a wide range of symptoms related to physical changes in the brain and includes Alzheimer's disease, which accounts for 60 to 80 percent of cases, and vascular dementia, which occurs after a stroke, which is the second most common dementia type, and senile dementia, which reflects the mental decline that is a normal part of aging, and also dementia with Lewy bodies (DLB), Frontotemporal dementia, dementia related to diseases such as Parkinson's disease, Creutzfeldt-Jakob disease, normal pressure hydrocephalus, Huntington's disease, Wernicke-Korsakoff Syndrome, and mixed dementia. In one embodiment the term “dementia” includes Alzheimer's disease, Vascular dementia, senile dementia, dementia with Lewy bodies (DLB), Frontotemporal dementia, dementia related to diseases such as Parkinson's disease, Creutzfeldt-Jakob disease, normal pressure hydrocephalus, Huntington's disease, Wernicke-Korsakoff Syndrome, and mixed dementia.

As used herein, the term “C₁₋₄alkyl”, used alone or as a suffix or prefix, is intended to include both branched and straight chain saturated aliphatic hydrocarbon groups having from 1 to 4 carbon atoms. Examples of C₁₋₄alkyl include methyl, ethyl, n-propyl, i-propyl, n-butyl, i-butyl, sec-butyl and tert-butyl.

As used herein, the term “C₁₋₄haloalkyl”, used alone or as a suffix or prefix, is intended to include both branched and straight chain saturated aliphatic hydrocarbon groups, having at least one halogen substituent, selected from fluoro, iodo, bromo and chloro, and having from 1 to 4 carbon atoms. Examples of C₁₋₄haloalkyl include, but are not limited to, fluoromethyl, difluoromethyl, trifluoromethyl, 1-fluoroethyl, difluoroethyl, trifluoroethyl, chloromethyl, chloroethyl, dichloroethyl, trifluoropropyl, bromomethyl, bromoethyl difluorobutyl and trifluorobutyl.

As used herein, the term “halogen” or “halo”, used alone or as suffix or prefix, is intended to include bromine, chlorine, fluorine and iodine.

The term “C₁₋₄haloalkyl-S—” refers to an alkylsulfanyl having at least one halogen atom. Exemplary halo-alkylsulfanyl includes fluoromethylsulfanyl, difluoromethylsulfanyl, trifluoromethylsulfanyl, fluoroethylsulfanyl and bromopropylsulfanyl.

The term “C₁₋₄haloalkyl-S(O)—” refers to an alkylsulfinyl having at least one halogen atom. Exemplary halo-alkylsulfinyl includes fluoromethylsulfinyl, difluoromethylsulfinyl, trifluoromethylsulfinyl fluoroethylsulfinyl and bromopropylsulfinyl.

The term “C₁₋₄haloalkyl-S(O)₂—” refers to an alkylsulfonyl having at least one halogen atom. Exemplary halo-alkylsulfonyl includes fluoromethylsulfonyl, difluoromethylsulfonyl, trifluoromethylsulfonyl fluoroethylsulfonyl and bromopropylsulfonyl.

As used herein, the term “optional” or “optionally” means that the subsequently described event or circumstance may but need not occur, and that the description includes instances where the event or circumstance occurs and instances where it does not.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to forms of the disclosed compounds, wherein the parent compound is modified by making acid or base salts thereof. Generally, pharmaceutically acceptable salts of the compound of the invention as defined above may be obtained using standard procedures well known in the art, for example by reacting a sufficiently basic compound, for example an alkyl amine with a suitable acid, for example, hydrochloride or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

The compound of the invention as defined above may be converted to a pharmaceutically acceptable salt or solvate thereof, particularly, an acid addition salt such as an alkali metal (such as sodium, potassium, or lithium) or an alkaline earth metal (such as a calcium) salt, a basic amine salt or a solvate thereof such as ammonia, amino acids (preferably histidine, lysine, ornithine), tetraalkyl ammonium salts (preferably carnitine and esters thereof, choline, tetraethyl ammonium, tetramethyl ammonium), aminopolyols (preferably tromethamine), purines, guanines, vitamins (preferably vitamins B1, B3, B6 and B1 1), amino sugars (preferably daunosamine, galactosamine, glucosamine, N-methylglucamine) and ethyl amine derivatives (preferably benzathine, diethyl amine, ethanol amine, ethyl amine, ethylene diamine, 1—(2-hydroxyethyl)-pyrrolidine, piperazine, triethanol amine, triethyl amine). In one embodiment, the pharmaceutically acceptable salt or solvate thereof, is a sodium salt or calcium salt.

A variety of compounds in the present invention may exist in particular geometric or stereoisomeric forms. The present invention takes into account all such compounds, including tautomers, R- and S- enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. All chiral, diastereomeric and racemic forms are intended, to be included in the scope of the invention, unless the specific stereochemistry or isomeric form is specifically indicated.

As used herein, “tautomer” means other structural isomers that exist in equilibrium resulting from the migration of a hydrogen atom. For example, keto-enol tautomerism occurs where the resulting compound has the properties of both a ketone and an unsaturated alcohol.

As used herein, the phrase “compounds or pharmaceutically acceptable salts” include hydrates and solvates thereof.

Compounds and salts described in this specification may be isotopically-labelled compounds (or “radio-labelled”). In that instance, one or more atoms are replaced by an atom having an atomic mass or mass number different from the atomic mass or mass number typically found in nature (i.e., naturally occurring). Examples of suitable isotopes that may be incorporated include ²H (also written as “D” for deuterium), ³H (also written as “T” for tritium), ¹¹C , ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁸Br, ⁷⁷Br, ¹²³I, ¹²⁴I, ¹²⁵I and ¹³¹I. The radionuclide that is used will depend on the specific application of that radio-labelled derivative. For example, for in vitro receptor labelling and competition assays, compounds that incorporate ³H or ¹⁴O are often useful. For radio-imaging applications ¹¹C or ¹⁸F are often useful. In some embodiments, the radionuclide is ³H. In some embodiments, the radionuclide is ¹⁴O. In some embodiments, the radionuclide is ¹¹O. And in some embodiments, the radionuclide is ¹⁸F.

For compounds of the invention containing a bicyclic heteroaromatic group in the R¹ position, it may be understood that the point of attachment of these groups to the triazine ring may be located at any position on the benzene or heterocyclic ring. However, it is preferred that the point of attachment is on the benzene ring (i.e. in the 4,5,6 or 7 (e.g. 5) position of the bicycle), resulting in a, for example, benzofuran-5-yl or indol-5-yl substituent.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the invention are included within the scope of the invention.

Furthermore, certain compounds of the invention may possess no or minimal pharmacological activity as such, but may be administered parenterally or orally, and thereafter be metabolised in the body to form other compounds of the invention that possess pharmacological activity as such.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

It is to be understood that the therapy or treatment and/or prevention may be for a human patient as well as for an animal, for example a dog, cat, horse, ape, etc. As used herein, the terms “mammals” and “patient” may include both humans and animals (particularly humans).

Another embodiment of the invention relates to a compound of formula I, COMPOUND 1 or, particularly, COMPOUND 2 or COMPOUND 3, as defined above, or any mixture thereof, or a pharmaceutically acceptable salt thereof, for use in prevention and/or treatment of cognitive disorders in animals, such as dogs, horses and cats.

Compounds of the present invention may be administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

The invention especially relates to use of the following compounds.

Compound 1

Generic name: Toltrazuril

IUPAC name: 1-methyl-3-(3-methyl-4-{4-[(trifluoromethyl)sulfanyl]phenoxy}phenyl)-1,3,5-triazinane-2,4,6-trione, and having structural formula

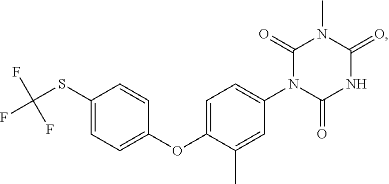 [SMILES: Cc1cc(-n2c(=O)[nH]c(=O)n(C)c2=O)ccc1Oc1ccc(SC(F)(F)F)cc1]

and/or

Compound 2

Generic name: Toltrazuril sulfone

IUPAC name: 1-methyl-3-[3-methyl-4-(4-trifluoromethanesulfonylphenoxy)phenyl]-1,3,5-triazinane-2,4,6-trione and having structural formula

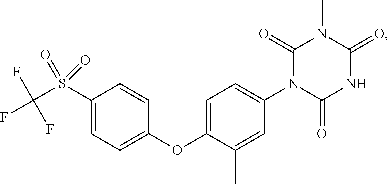 [SMILES: Cc1cc(-n2c(=O)[nH]c(=O)n(C)c2=O)ccc1Oc1ccc(S(=O)(=O)C(F)(F)F)cc1]

and/or

Compound 3

Generic name: Toltrazuril sulfoxide

IUPAC name: 1-methyl-3-[3-methyl-4-(4-trifluoromethanesulfinylphenoxy)phenyl]-1,3,5-triazinane-2,4,6-trione and having structural formula

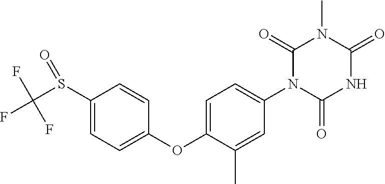 [SMILES: Cc1cc(-n2c(=O)[nH]c(=O)n(C)c2=O)ccc1Oc1ccc(S(=O)C(F)(F)F)cc1]

as modulators for neurotrophin receptors, such as TrkA, TrkB, TrkC and/or their signalling and receptor tyrosine kinases, such as FGFR1 and IGF1R and/or their signalling in the treatment and/or prevention of both non-neurological and neurological diseases, or any one of the diseases mentioned above, especially Alzheimer's disease, Parkinson's disease, Cognitive dysfunction in Schizophrenia, Rett's Syndrome and Depression.

### Preparation of Compounds

Compounds of the invention can be prepared as a free base or a pharmaceutically acceptable salt thereof by processes described in various patents. For example, in U.S. Pat. No. 4,219,552, which is hereby included by reference, especially examples 2, 3 on column 10 and example 15 on column 11 of U.S. Pat. No. 4,219,552.

### BRIEF DESCRIPTION OF THE FIGURES

FIG. 1 shows the results of the passive avoidance task described in Example 1. The graph demonstrates that administering COMPOUND 1 to mice treated with scopolamine improves cognitive function, as illustrated by the increased retention latency within the bright area.

FIG. 2 shows the results of the passive avoidance task described in Example 2. The graph demonstrates that administering COMPOUND 2 to mice treated with MK-801 improves cognitive function, as illustrated by increased time spent in the bright area.

FIG. 3 shows a comparison of the effects of COMPOUNDS 1, 2 and 3 on TrkB expressing cells stimulated with or without brain-derived neurotrophic factor (BDNF). COMPOUND 1 (toltrazuril) demonstrates no clear dose-response curve when BDNF is used a ligand using cells expressing the BDNF receptor TrkB. Furthermore, the activity of COMPOUND 1 significantly decreases at the two highest concentrations tested (A1, B1 and C1). This effect is surprisingly not observed for COMPOUND 2 (Toltrazuril sulfone) (A2, B2 and C2) or COMPOUND 3 (Toltrazuril sulfoxide) (A3, B3 and C3).

FIG. 4 shows a comparison of the effects of COMPOUNDS 1, 2 and 3 on TrkA expressing cells stimulated with or without NGF. A similar trend to the experiment with TrkB and BDNF is observed for toltrazuril (A1, A2 and A3), although the results are not statistically significant. This indicates that the decreased capacity of Toltrazuril compared to its oxidised derivatives to activate TrkB is not due to general cytotoxicity, but rather due to the specific pharmacological action of the compounds.

### EXAMPLES

### General Methods

All solvents were of analytical grade and commercially available anhydrous solvents were routinely used for reactions. Starting materials used were available from commercial sources or prepared according to literature procedures, Room temperature referes to 20-25° C. Solvent mixture compositions are given as volume percentages or volume ratios.

MW heating was performed in a standard MW reactor producing continuous irradiation at 2450 MHz. It is understood that MWs can be used for the heating of reaction mixtures.

Thin layer chromatography (TLC) was performed on Merck TLC-plates (Silica gel 60 F₂₅₄) and spots were UV visualized. TLC was generally used to monitor reaction progression and solvents used were for example: ethyl acetate or acetonitrile or DCM with 1-10% of MeOH, ethyl acetate with 0-95% hexane. Straight phase flash column chromatography (“flash chromatography”/“column chromatography”) was manually performed on Merck Silica gel 60 (0.040-0.063 mm) or basic aluminum oxide or neutral aluminum oxide, or automatically using ISCO Combiflash® Companion™ system using RediSep™ normal-phase flash columns (“Combiflash”) using the solvent system indicated.

### NMR

NMR spectra was recorded on a 400 MHz NMR spectrometer (Bruker 400 MHz Avance-III) fitted with a probe of suitable configuration. Spectra were recorded at ambient temperature unless otherwise stated. Chemical fields are given in ppm down- and upfield from TMS (0.00 ppm). The following reference signals were used in ¹H-NMR: TMS ∂ 0.00, or residual solvent signal of DMSO-d6 δ 2.49, CDCl₃δ 7.25 (unless otherwise indicated). Resonance multiplicities are denoted s, d, t, q, m, dd, tt, dt br and app for singlet, doublet, triplet, quartet, doublet of doublet, triplet of triplet, doublet of triplet, multiplet, broad and apparent, respectively. In some cases only diagnostic signals are reported.

### HPLC, HPLCMS and LCMS Analysis

High pressure liquid chromatography (HPLC) was performed on a reversed phase (RP) column. A gradient was applied using for example mobile phase A (5 mM Ammonium acetate +0.1% Formic acid in water) and B (0.1% Formic acid in Acetonitrile) or A (0.1% NH3 in water) and B (0.1% NH3 in acetonitrile) or A (10 mM Ammonium actetate in water) and B (Acetonitrile).

Reversed phase columns used were for example: BEH C18 (50*2.1 mm), 1.7 μm; X-Bridge C18 (50*4.6 mm), 3.5 μm; X-Bridge/YMCC18 (150*4.6 mm), 5 μm; BEH C18 (50*2.1 mm), 1.7 μm. The flowrate used was for example 0.55 ml/min or 1.00 ml/min Mass spectrometry (MS) analysis were performed in positive and/or negative ion mode using electrospray ionization (ESI+/−).

### Preparative HPLC Chromatography

Preparative chromatography was run on a Waters e2695 Separation Module with a PDA Detector. Column; X-BRIDGE C18, 150*4.6 mm, 5μm or X-Bridge C18 (250*19 mm) 5μm or GEMINI C18 (250*21.2 mm) 5μm.

A gradient was applied using for example mobile phase A (0.1% NH₃ in water) and B (0.1% NH3 in acetonitrile); A (0.1% TFA in water) and B (Acetonitrile); A (5 mM ammonium bicarbonate+0.05% ammonia in water) and B (Acetonitrile); A (5 mM ammonium bicarbonate) and B (acetonitrile) for LC-separation at a flow rate 1 ml/min.

### Straight (Normal) Phase HPLC Analysis

High pressure liquid chromatography (HPLC) was performed on a straight (normal) phase column. A linear gradient or isocratic flow was applied using for example phase A (Hexane) and B (XX)

### Abbreviations

DCM dichloromethane
DMSO dimethylsulfoxide
TEA triethylamine

Compounds have been named using CDD vault from Collaborative Drug Discovery Inc. Burlingame Calif., USA or ChemDoodle 8.1.0 from iChemLabs LLC, USA or ACD/ChemSketch 2012 (14.01) from Advanced Chemistry Development (ACD/labs) Ontario, Canada. In case of inconsistency between a name of a compound and the structural formula of the same compound, it is the structural formula that is decisive for the molecular structure of the compound.

### Intermediate 1

1-(3-methyl-4-{4-[(trifluoromethyl)sulfanyl]phenoxy}phenyl)-3-phenylurea

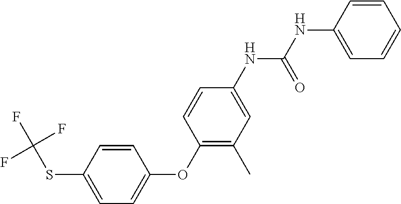 [SMILES: Cc1cc(NC(=O)Nc2ccccc2)ccc1Oc1ccc(SC(F)(F)F)cc1]

3-methyl-4-{4-[(trifluoromethyl)sulfanyl]phenoxy}aniline (commercially available, 1.78 g, 0.0059 mol) and TEA (1.67 mL) were added to DCM (18.0 mL) and stirred and cooled to 0° C. Phenyl isocyanate (0.708 mL, 0.0065 mol) was added and the reaction mixture was stirred at 25° C. for 16 h. The solvent was evaporated and the reaction mixture was quenched with water (25 ml) and product was extracted with ethyl acetate (3×30 ml). The combined organic layers were washed with brine (30 ml), dried over sodium sulfate and evaporated under reduced pressure to obtain the crude product. The crude product was purified on column chromatography by 15% ethyl acetate in hexane as a mobile phase and 100-200 silica as stationary phase to yield 2.25 g (90% yield) of the title compound. ¹H NMR (400 MHz, DMSO-d6) δ ppm 2.09 (s, 3H) 6.87-7.12 (m, 4H) 7.18-7.41 (m, 4H), 7.41-7.51 (m, 4H) 7.64-7.72 (s, 1H) 8.76 (s, 1H); MS (ES+) m/z 419 [M+H]⁺

### Intermediate 2

1-(3-methyl-4-{4-[(trifluoromethyl)sulfinyl]phenoxy}phenyl)-3-phenylurea

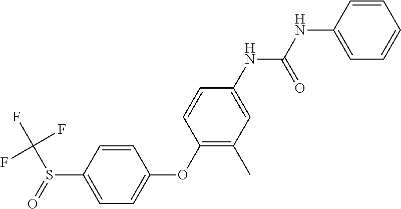 [SMILES: Cc1cc(NC(=O)Nc2ccccc2)ccc1Oc1ccc(S(=O)C(F)(F)F)cc1]

Oxone (8.810 g, 0.0143 mol) was added portion-wise to a solution of 1-(3-methyl-4-{4-[(trifluoromethyl)sulfanyl]phenoxy}phenyl)-3-phenylurea (Intermediate 68, 1.5 g, 0.0035 mol) in methanol (30 mL) at 25° C. and stirred for 48 hours at same temperature. The reaction mixture was quenched with ice-water (50 ml) and extracted with ethyl acetate (3×40 ml). The combined organic layers were washed with brine (30 ml), dried over sodium sulfate and evaporated under reduced pressure to obtain the crude product. The crude product was purified on silica gel (100-200 mesh) using 60% ethyl acetate in hexane as an eluent to yield 0.580 g (39% yield) of the title compound. ¹H NMR (400 MHz, DMSO-d6) δ ppm 2.10 (s, 3H) 6.92-7.00 (m, 1H) 7.02-7.07 (m, 1H) 7.09-7.17 (m, 2H) 7.23-7.30 (m, 2H) 7.32-7.41 (m, 1H) 7.43-7.53 (m, 3H) 7.83-7.90 (m, 2H) 8.69 (s, 1H) 8.73 (s, 1H); MS (ES-) m/z 433 [M-H]

### Synthetic Example 1 (COMPOUND 4)

### 1-[3-methyl-4-(4-trifluoromethanesulfinylphenoxy)phenyl]-3-phenyl-1,3,5-triazinane-2,4,6-trione

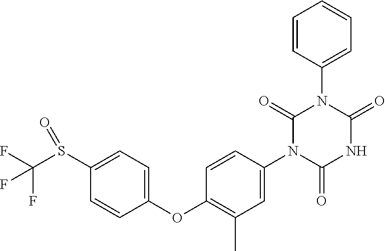 [SMILES: Cc1cc(-n2c(=O)[nH]c(=O)n(-c3ccccc3)c2=O)ccc1Oc1ccc(S(=O)C(F)(F)F)cc1]

In a 30 ml seal tube previously equipped with a magnetic stirrer, ethoxycarbonyl isocyanate (0.291 g, 0.0025 mol) was added to a solution of 1-(3-methyl-4-{4-[(trifluoromethyl)sulfinyl]phenoxy}phenyl)-3-phenylurea (Intermediate 69, 0.570 g, 0.0012 mol) in toluene (5.70 mL) under stirring at 0° C. The resulting reaction mixture was heated at 110° C. for 16 h. The solvent was evaporated under reduced pressure to obtain the crude product. The crude product was purified by preparative HPLC purification using 0.1% ammonia as a modifier and water:acetonitrile (0-100% gradient system) as a mobile phase to yield 0.045 g (8% yield) of the title compound. ¹H NMR (400 MHz, DMSO-d6) δ ppm 2.18 (s, 3 H) 7.15-7.26 (m, 3 H) 7.29-7.36 (m, 1 H) 7.37-7.55 (m, 6 H) 7.90-7.98 (m, 2 H) 12.05 (s, 1 H); MS (ES-) m/z 502 [M-H]⁻

### Synthetic Example 2 (COMPOUND 5)

### 1-(3-methyl-4-{4-[(trifluoromethyl)sulfanyl]phenoxy}phenyl)-3-phenyl-1,3,5-triazinane-2,4,6-trione

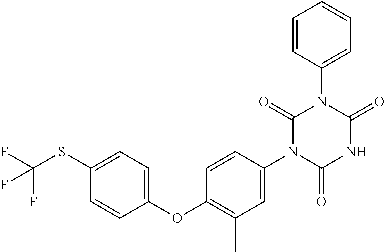 [SMILES: Cc1cc(-n2c(=O)[nH]c(=O)n(-c3ccccc3)c2=O)ccc1Oc1ccc(SC(F)(F)F)cc1]

In a 30 ml seal tube previously equipped with a magnetic stirrer, 1-(3-methyl-4-{4-[(trifluoromethyl)sulfanyl]phenoxy}phenyl)-3-phenylurea (Intermediate 68, 0.60 g, 0.0014 mol) was dissolved in toluene (6.00 mL) and cooled to 0° C. To the reaction mixture, ethoxycarbonyl isocyanate (0.247 g, 0.0021 mol) was added. The reaction mixture was stirred at 110° C. for 16 h. The solvent was evaporated under reduced pressure to obtain the crude product. The crude product was purified on combi flash chromatography by using ethyl acetate as a mobile phase and 60-120 silica as stationary phase. The obtained product was further purified by preparative HPLC purification using 5 mM ammonium bicarbonate +0.05% ammonia as modifier and water:acetonitrile (0-100% gradient system) as a mobile phase to yield 0.020 g (2.9% yield) of the title compound. ¹H NMR (400 MHz, DMSO-d6) δ ppm 2.15 (s, 3 H) 6.99-7.06 (m, 2 H) 7.08-7.14 (m, 1 H) 7.24-7.30 (m, 1 H) 7.33-7.51 (m, 6 H) 7.68-7.75 (m, 2 H) 12.00 (s, 1 H); MS (ES-) m/z 486 [M-H]³¹

### Synthetic Example 3 (COMPOUND 6)

### 1 -[3-methyl -4-(4-trifl uoromethanesulfonylphenoxy)phenyl]-3-phenyl-1,3,5-triazinane-2,4,6-trione

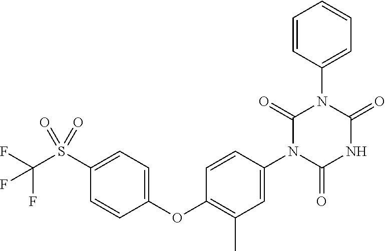 [SMILES: Cc1cc(-n2c(=O)[nH]c(=O)n(-c3ccccc3)c2=O)ccc1Oc1ccc(S(=O)(=O)C(F)(F)F)cc1]

m-Chloroperoxybenzoic acid (70%, 0.048 g, 0.000198 mol) was added portion-wise to a solution of 1-[-3-methyl-4-(4-trifluoromethanesulfinylphenoxy)phenyl]-3-phenyl-1,3,5-triazinane-2,4,6-trione (Synthetic Example 1 (COMPOUND 4), 0.025 g, 0.0000496 mol) in DCM (0.250 ml) at 0° C. The reaction mixture was stirred for 16 h at 25° C. The reaction mixture was quenched with ice-water (20 ml) and extracted with DCM (3×10 ml). The combined organic layer was washed with brine (10 ml), dried over sodium sulfate and concentrated under reduced pressure. The crude product was purified using preparative HPLC by using 0.1% ammonia as modifier and water:acetonitrile (0-100% gradient system) as a mobile phase to yield 0.011 g (42% yield) of the title compound. ¹H NMR (400 MHz, DMSO-d6) δ ppm 2.16 (s, 3H) 7.22-7.33 (m, 3H) 7.33-7.55 (m, 7H) 8.15 (s, 1H) 8.17 (s, 1H) 12.08 (s, 1H); MS (ES-) m/z 518 [M-H]⁻

### BIOLOGICAL ASSAYS

### In Vitro Assay

A high throughput cell-based screen has been used to identify positive modulators of TrkA, TrkB and TrkC. The screen involves the use of cell-based assay overexpressing TrkA, TrkB or TrkC. The purpose of the assay is to identify compounds that modulate neurotrophin signalling (Forsell et al 2012). The assay can be used in inhibitor mode using a high concentration of ligand, in modulator mode using an intermediate concentration and in agonist mode using a low concentration of ligand.

The assay uses Enzyme Fragment Complementation (EFC) technique, which is a proximity-based assay. Briefly, cells used in this assay over-express two fusion proteins, i.e. the receptor, which can be either one of TrkA, TrkB, TrkC, IGF1R or FGFR1, fused to a small peptide of 8-galactosidase and an adaptor protein, i.e. SHC1 (or any other Trk-adaptor protein) fused to the major part of 8-galactosidase. Ligand binding to the receptor induces phosphorylation of the intracellular domain and hence, recruitment of the adaptor protein to the receptor. The proximity between the small activating peptide on the receptor and the major part of 8-galactosidase on the adaptor protein leads to an active 8-galactosidase enzyme. The activation of the receptor is quantified by measuring the amount of active 8-galactosidase by its conversion of a non-luminescent substrate into a luminescent product.

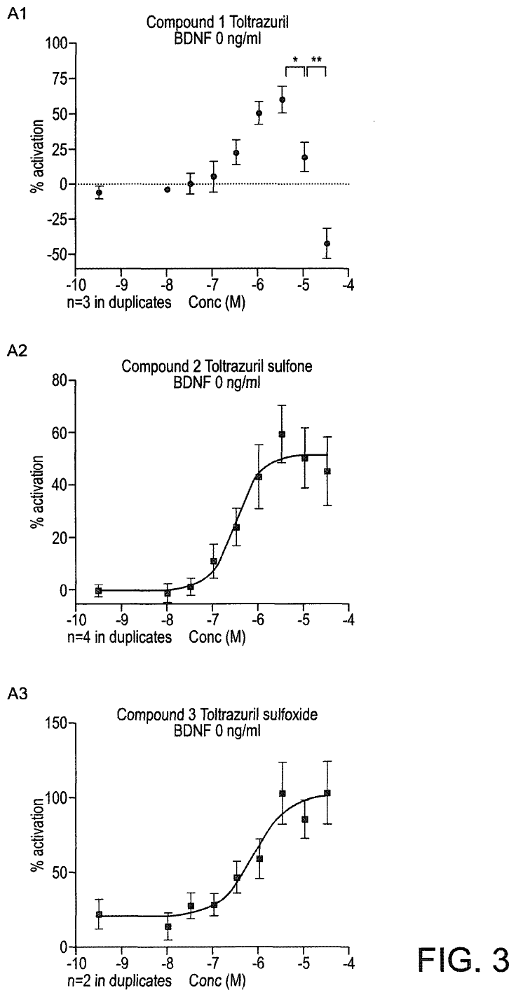

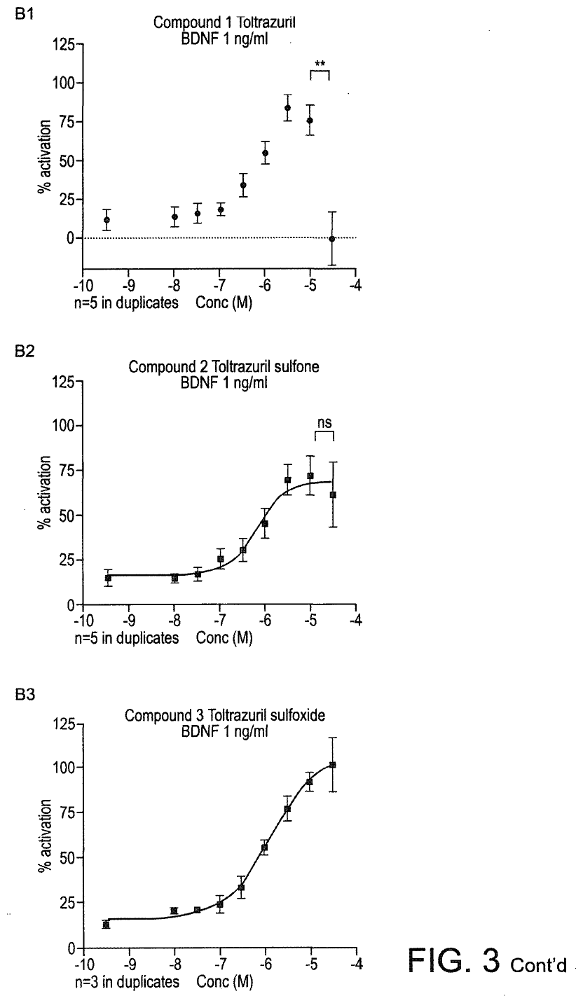

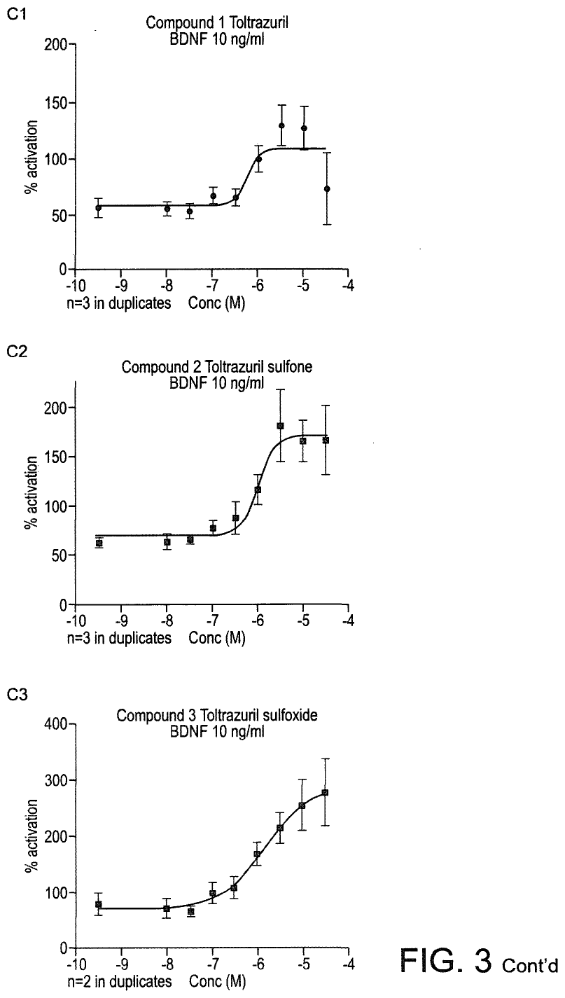

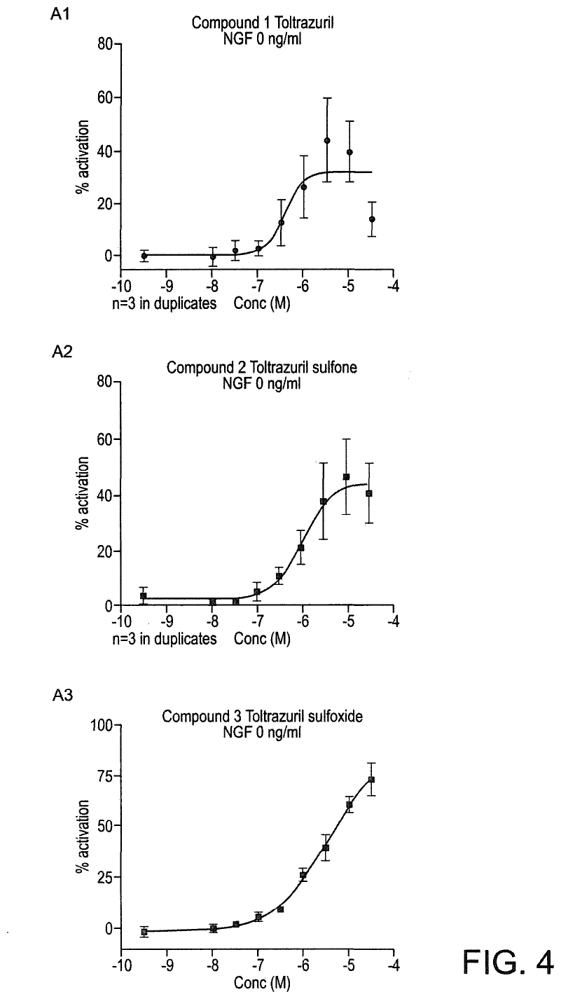

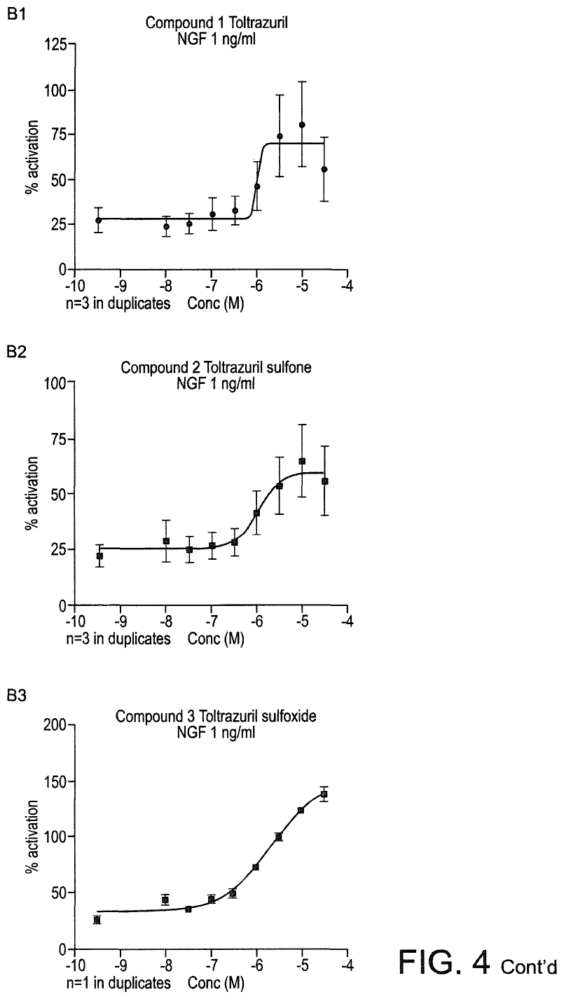

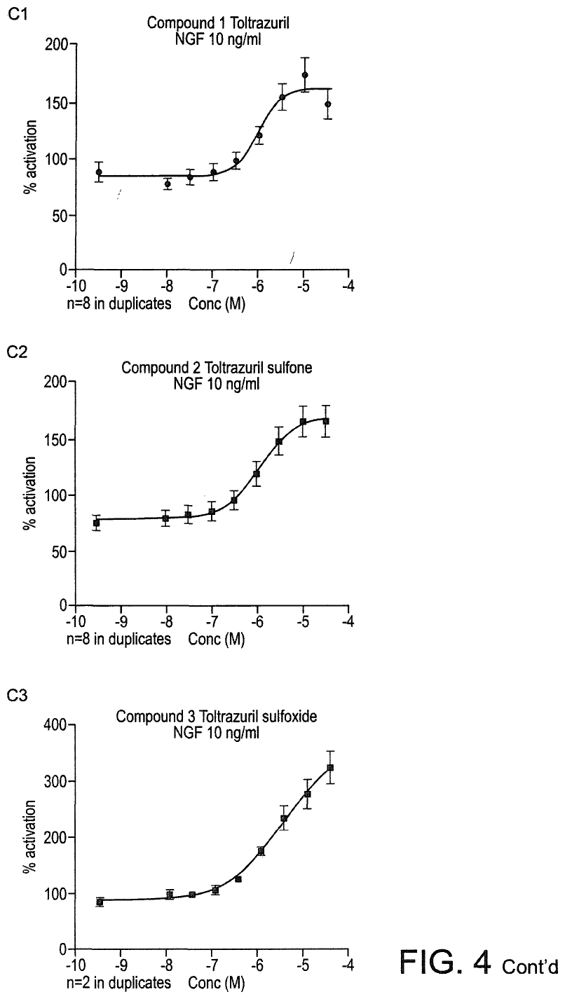

U2OS-cells, over-expressing TrkA or TrkB or TrkC, are plated in 96- or 384-well plates and incubated overnight. Alternatively, cryopreserved HEK293-cells expressing IGFR1 or cryopreserved U2OS-cells expressing FGFR1 were plated in 96- or 384-well plates. On the following day, test compound was pre-mixed with ligand (NGF, BDNF, NT-3, IGF-1 or basic fibroblast growth factor (bFGF(FGF-2))) and the ligand-compound mixture is then added to the cells to yield a final ligand concentration of typically 10 ng/mL (or as indicated in FIGS. 3 and 4). After 3 hours of incubation at room temperature, the incubation is stopped by the addition of a β-galactosidase substrate mixture containing detergents. The substrate mixture is incubated for 60 minutes at ambient temperature. The luminescence is thereafter read by the use of a plate reader.

### In Vivo Assay

Passive Avoidance task

Passive avoidance (PA) is an aversive learning task based on classical (Pavlovian) fear conditioning that allows for analysis of both facilitation and impairment of memory function by adjusting the unconditioned stimulus, i.e. the electrical foot shock. Commonly a cognitive-impairing agent is administered to the animals to mimic the neurochemical disturbances present in various cognitive disorders e.g. cholinergic (scopolamine) and glutamatergic (MK-801) deficits.

Prior to testing, the animals are brought to the experimental room where they were allowed to habituate for 60 min. The test is conducted using a modified shuttle box with two communicating compartments of equal size with a small sliding door built into the separating wall and a stainless steel bar floor. One of the compartments is not illuminated and thus black whereas the other compartment (the light one) is illuminated by an electric bulb, installed on the top of a plexiglass cover. The PA training is conducted in a single session. The animals are allowed to explore the compartment for 60 sec, after which the sliding door is automatically opened and the mouse is allowed to cross over into the dark compartment. Once the mouse has entered the dark chamber with all four feet, the sliding door is automatically closed and a scrambled electrical current is delivered through the grid floor. Latency to cross over into the dark compartment (training latency) is recorded. Retention latencies as well as total time spent in bright compartment are tested 24 h later (day two). The animals are placed in the light compartment and allowed to explore for 15 sec, where upon the sliding door is opened allowing free access to the dark compartment for a period of 300 sec. The latency to cross over into the dark compartment with all four feet is measured (retention latency) as well as time in bright compartment and a number of other relevant parameters (e.g. number of visits in the dark compartment).

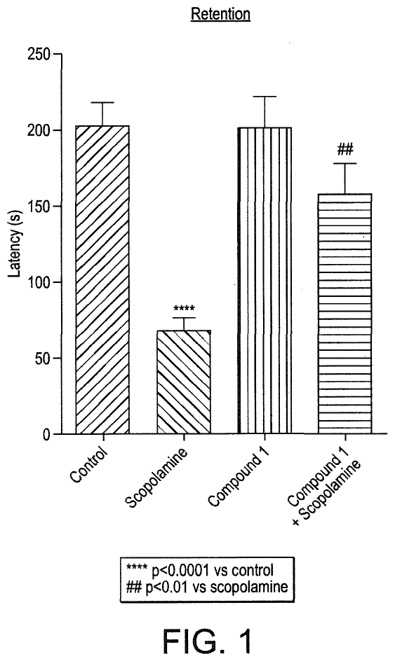

In Example 1, vehicle (20 % DMSO in 0.1M PBS) or Compound 1 (20 mg/kg) was administered to C57/BI6 mice once per day (i.p. administration) for 4 days prior to PA training. Moreover, on the day of PA training, scopolamine at 0.3 mg/kg, or vehicle, was administered subcutaneously 30 min prior to training. Data on retention latency shown in FIG. 1.

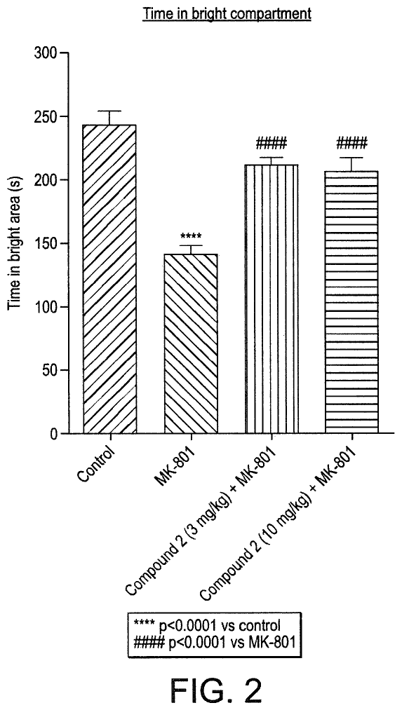

In Example 2, vehicle (20 % DMSO in 0.1 M PBS) or different doses of Compound 2 was administered to C57/BI6 mice once per day (i.p administration) for 4 days prior to PA training. Moreover, on the day of PA training, MK-801 at 0.3 mg/kg, or vehicle, was administered subcutaneously 30 min prior to training. Data on total time spent in bright compartment shown in FIG. 2.

In Example 3, vehicle (20 % DMSO in 0.1 M PBS) or different doses of Compound 3 is administered to C57/BI6 mice once per day (i.p administration) for 4 days prior to PA training. Moreover, on the day of PA training, MK-801 at 0.3 mg/kg, or vehicle, was administered subcutaneously 30 min prior to training.

### Results (In Vitro Assay)

Data from these assays for representative compounds is shown in the Table below. The potency is expressed as EC50 (pM) and the efficacy as % stimulation over 10 ng/mL of ligand . The ligands used was: NGF (for TrkA assay), BDNF (for TrkB assay), NT-3 (for TrkC assay) bFGF (for FGFR1 assay), IGF-1 (for IGFR1 assay), respectively. The data indicate that the compounds of the invention are expected to possess useful therapeutic properties.

| Compound | TrkA | TrkB | TrkC | FGFR1 | IGF1R |
|---|---|---|---|---|---|
| 1 | 1.2 (109%) | 0.85 (124%) | 3.4 (87%) | 0.42 (73%) | 0.68 (83%) |
| 2 | 1.7 (138%) | 2.5 (232%) | 0.66 (70%) | 0.84 (43%) | 0.92 (25%) |
| 3 | 2.9 (143%) | 3.3 (196%) | 0.77 (74%) | 0.66 (102%) | 1.5 (109%) |
| 4 | 1.4 (128%) | 2.3 (217%) | 0.85 (69%) | | |
| 5 | 0.7 (112%) | 1.3 (210%) | 0.66 (84%) | | |
| 6 | 0.87 (147%) | 1.76 (300%) | 0.97 (124%) | | |

1. (canceled)

2. A method of treating and/or preventing a disease characterised by impaired signalling of neurotrophins and/or other trophic factors in a patient with the Val66Met mutation in the brain-derived neurotrophic factor gene, the method comprising:
administering to a patient in need thereof a therapeutically effective amount of a compound of formula I 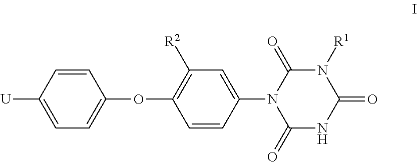
wherein:
R¹ represents phenyl optionally substituted by one or more groups selected from
C₁₋₄ alkyl, —OC₁₋₄alkyl, halogen, —OC₁₋₄haloalkyl or methylenedioxy; thiophenyl optionally substituted by one or more methyl groups; benzofuranyl; indolyl; or
R² represents —OC₁₋₄alkyl optionally substituted by one or more methoxy groups; or C₁₋₄alkyl; and
U is selected from the group consisting of C₁₋₄haloalkyl-S—, C₁₋₄haloalkyl-S(O)—, and C₁₋₄haloalkyl-S(O)₂—,
or a pharmaceutically-acceptable salt or prodrug thereof.

3. (canceled)

4. The method as claimed in claim 2, wherein R¹ represents phenyl optionally substituted by one or more groups selected from C₁₋₂alkyl, —OC₁₋₂alkyl, Cl, F, —OC₁₋₂haloalkyl or methylenedioxy; thiophenyl optionally substituted by one or more methyl groups; benzofuranyl; indolyl; or C₁₋₄alkyl.

5. The method as claimed in claim 2, wherein R¹ represents phenyl optionally substituted by one group selected from methyl, methoxy, Cl, F, —OCF₃ or methylenedioxy; or C₁₋₄alkyl.

6. The method as claimed in claim 2, wherein R² represents C₁₋₂alkyl; or —OC₁₋₃alkyl optionally substituted by one or more methoxy groups.

7. The method as claimed in claim 2, wherein R² represents methyl, methoxy, ethoxy, iso-propoxy or —OCH₂CH₂OCH₃.

8. The method as claimed in claim 2, wherein U is selected from the group consisting of CF₃S—, CF₃S(O)— and CF₃S(O)₂—.

9. The method as claimed in claim 2, wherein
R¹ represents methyl or phenyl,
R² represents C₁₋₂alkyl, and
U is selected from the group consisting of C₁₋₂fluoroalkyl-S—, C₁₋₂fluoroalkyl-S(O)— and C₁₋₂fluoroalkyl-S(O)₂—.

10. The method as claimed in claim 9, wherein the compound is 1-methyl-3-(3-methyl-4-{4-[(trifluoromethyl)sulfanyl]phenoxy}phenyl)-1,3,5-triazinane-2,4,6-trione or a pharmaceutically acceptable salt or prodrug thereof.

11. (canceled)

12. A method of treating and/or preventing a disease characterised by impaired signalling of neurotrophins and/or other trophic factors, comprising:
administering to a patient in need thereof a therapeutically effective amount of a compound of formula I, 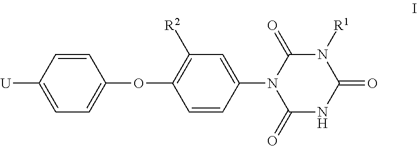
wherein:
R¹ represents phenyl optionally substituted by one or more groups selected from
C₁₋₄alkyl, —OC₁₋₄alkyl, halogen, —OC₁₋₄haloalkyl or methylenedioxy; thiophenyl optionally substituted by one or more methyl groups; benzofuranyl; indolyl; or
R² represents —OC₁₋₄alkyl optionally substituted by one or more methoxy groups; or C₁₋₄alkyl; and
U is selected from the group consisting of C₁₋₄haloalkyl-S(O)— and C₁₋₄haloalkyl-S(O)₂—. or a pharmaceutically acceptable salt or prodrug thereof.

13. (canceled)

14. The method as claimed in claim 12, wherein
R¹ represents methyl or phenyl,
R² represents C₁₋₂alkyl, and
U is selected from the group consisting of C₁₋₂fluoroalkyl-S(O)— and C₁₋₂fluoroalkyl-S(O)₂—.

15. The method as claimed in claim 12, wherein
R¹ represents methyl,
R² represents C₁₋₄alkyl, and
U is selected from the group consisting of C₁₋₂fluoroalkyl-S(O)— and C₁₋₂fluoroalkyl-S(O)₂—.

16. The method as claimed claim 12, wherein the compound is
1-methyl-3-[3-methyl-4-(4-trifluoromethanesulfonylphenoxy)phenyl]-1,3,5-triazinane-2,4,6-trione,
1-methyl-3-[3-methyl-4-(4-trifluoromethanesulfinylphenoxy)phenyl]-1,3,5-triazinane-2,4,6-trione, or
a pharmaceutically acceptable salt or prodrug thereof.

17. A compound of formula I, as defined in claim 1, 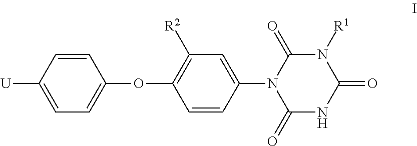
wherein
R¹ represents C₂₋₄alkyl; phenyl optionally substituted by one or more groups selected from C₁₋₄alkyl, —OC₁₋₄alkyl, halogen, —OC₁₋₄haloalkyl or methylenedioxy; thiophenyl optionally substituted by one or more methyl groups; benzofuranyl; or indolyl,
R² represents —OC₁₋₄alkyl optionally substituted by one or more methoxy groups; or C₁₋₄alkyl; and
U is selected from the group consisting of C₁₋₄haloalkyl-S—, C₁₋₄haloalkyl-S(O)—, and C₁₋₄haloalkyl-S(O)₂—, or a pharmaceutically acceptable salt or prodrug thereof.

18. A compound as claimed in claim 17, wherein R represents C₂₋₄alkyl; phenyl optionally substituted by one or more groups selected from C₁₋₂alkyl, —OC₁₋₂alkyl, Cl, F, —OC₁₋₂haloalkyl or methylenedioxy; thiophenyl optionally substituted by one or more methyl groups; benzofuranyl; or indolyl.

19. A compound as claimed in claim 17, wherein R¹ represents phenyl optionally substituted by one group selected from methyl, —OCH₃, Cl, F, —OCF₃ or methylenedioxy.

20. A compound as claimed in claim 17, wherein R² represents methyl, methoxy, ethoxy, iso-propoxy or —OCH₂CH₂OCH₃.

21. A compound as claimed in claim 17, wherein U is selected from the group consisting of CF₃S—, CF₃S(O)— and CF₃S(O)₂—.

22. A compound as claimed in claim 17, wherein
R¹ represents C₂₋₄alkyl or phenyl;
R² represents C₁₋₂alkyl; and
U is selected from the group consisting of C₁₋₂fluoroalkyl-S—, C₁₋₂fluoroalkyl-S(O)— and C₁₋₂fluoroalkyl-S(O)₂—.

23. A compound as claimed in claim 17, wherein
R₁ resents phenyl;
R² represents methyl; and
U is selected from the group consisting of CF₃S—, CF₃S(O)— and CF₃S(O)₂—.

24. (canceled)

25. The method as claimed in claim 2, wherein the disease characterised by impaired signalling of neurotrophins and/or other trophic factors is selected from the group consisting of Alzheimer's disease, depression, Parkinson's disease, other Parkinsonian disorders, other tauopathies, Lewy body dementia, multiple sclerosis, Huntington's disease, mild cognitive impairment, brain injuries, stroke, other dementia disorders, motorneurone diseases, Pick disease, spinal chord injury, hypoxic ischemia injury, cognitive dysfunction, coronary artery disease, obesity, metabolic syndrome, diabetes, Charcot-Marie-Tooth disease, diabetic neuropathy, tissue regeneration, motor function, nerve injury, hearing loss, blindness, posterior eye diseases, dry eye disease, neurotrophic keratitis, glaucoma, high intraocular pressure, retinitis pigmentosa, post-traumatic stress disorders, WAGR syndrome, diseases of the olfactory tract, olfactory decline, olfactory dysfunction, anxiety, fragile X syndrome, congenital central hypoventilation syndrome, obsessive-compulsive disorder, generalized anxiety disorder, eating disorders, bipolar disorder, chronic fatigue syndrome, neuromyelitis optica, Rett syndrome, Friedrich's ataxia and obstructive sleep apnea-hypopnea syndrome.

26. The method as claimed in claim 25, wherein the disease characterised by impaired signalling of neurotrophins and/or other trophic factors is selected from the group consisting of Alzheimer's disease, Parkinson's disease, other Parkinsonian diseases, other tauopathies, Lewy body dementia, motorneuron disease, Pick disease, obesity, metabolic syndrome, diabetes and Rett Syndrome.

27. The method or use as claimed in claim 25, wherein the disease characterised by impaired signalling of neurotrophins and/or other trophic factors is selected from the group consisting of Alzheimer's disease, Parkinson's disease, cognitive dysfunction, depression and Rett syndrome.

28. The method as claimed in claim 25, wherein the disease characterised by impaired signalling of neurotrophins and/or other trophic factors is Alzheimer's disease.

29. A pharmaceutical composition comprising a compound as defined in claim 17, or a pharmaceutically-acceptable salt or prodrug thereof, and optionally a pharmaceutically acceptable adjuvant, diluent or carrier.

30. (canceled)

31. A combination product comprising:
(I) a compound as defined in claim 17 or a pharmaceutically acceptable salt or prodrug thereof; and
(II) one or more other therapeutic agent that is useful in the treatment or prevention of a disease characterised by impaired signalling of neurotrophins and/or other trophic factors, wherein each of components (I) and (II) is fomulated in admixture, optionally with a pharmaceutically acceptable adjuvant diluent or carrier.

32. A kit-of-parts comprising:
(a) a pharmaceutical composition as defined in claim 29; and
(b) a pharmaceutical composition comprising one or more other therapeutic agent that is useful in the treatment or prevention of a disease characterised by impaired signalling of neurotrophins and/or other trophic factors, optionally in admixture with one or more pharmaceutically-acceptable excipient,
which components (a) and (b) are each provided in a form that is suitable for administration in conjunction with the other.

33. A process for the preparation of a compound as defined in claim 17, or a pharmaceutically acceptable salt thereof, comprising the step of reacting a compound of formula II 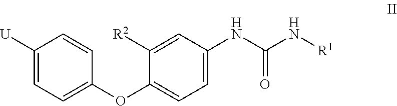
wherein R¹, R² and U are as defined in claim 17,
with a compound of formula III 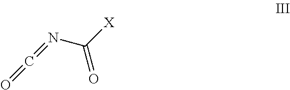
wherein X represents a suitable leaving group,
in the presence of a suitable solvent.

34. A process for the preparation of a pharmaceutical composition comprising bringing into association a compound as defined in claim 17, or a pharmaceutically acceptable salt or prodrug thereof, with a pharmaceutically acceptable adjuvant, diluent or carrier.
